(19) United States
(12) Patent Application Publication
(10) Pub. No.: US 2023/0226208 A1
(43) Pub. Date: Jul. 20, 2023

(54) METHODS FOR THE PREPARATION OF BIOCONJUGATES

(71) Applicant: Synaffix B.V., Oss (NL)

(72) Inventors: Maria Antonia WIJDEVEN, Oss (NL); Remon VAN GEEL, Lithoijen (NL); Laureen DE BEVER, Oss (NL); Sander Sebastiaan VAN BERKEL, Oss (NL); Floris Louis VAN DELFT, Nijmegen (NL)

(21) Appl. No.: 18/116,172

(22) Filed: Mar. 1, 2023

Related U.S. Application Data
(63) Continuation of application No. PCT/EP21/74296, filed on Sep. 2, 2021.

(30) Foreign Application Priority Data
Sep. 2, 2020 (NL) 2026400

(51) Int. Cl. A61K 47/68

(52) U.S. Cl. CPC A61K 47/6803; A61K 47/6889

(57) ABSTRACT

A surfactant for a bioconjugation reaction is disclosed, wherein a biomolecule comprising one or more azide moieties is connected to an cyclic alkyne comprising payload. More specifically, the invention concerns a method for the preparation of a bioconjugate of structure B—(Z—L—D)ₓ (1), comprising reacting (i) an alkyne or alkene compound of structure Q—L—D (2), wherein Q is a click probe comprising a cyclic alkyne moiety or a cyclic alkene moiety, L is a linker, and D is a payload; with (ii) a molecule of structure B—(F)ₓ (3), wherein B is a biomolecule that is functionalized with x click probes F; F is a click probe capable of reacting with Q, and x is an integer in the range of 1-10, in presence of a surfactant.

### CROSS-REFERENCE TO RELATED APPLICATIONS

This application is a continuation of International Patent Application No. PCT/EP2021/074296 filed Sep. 2, 2021, which application claims priority to European Patent

Application No. 2026400 filed September 2, 2020, the contents of which are both incorporated herein by reference in their entireties.

### FIELD OF THE INVENTION

The present invention relates to the field of bioconjugation, in particular to a method for preparing bioconjugates in the presence of surfactants.

### BACKGROUND OF THE INVENTION

Antibody-drug conjugates (ADC), considered as magic bullets in therapy, are comprised of an antibody to which is attached a pharmaceutical agent. The antibodies (also known as ligands) can be small protein formats (scFv's, Fab fragments, DARPins, Affibodies, etc.) but are generally monoclonal antibodies (mAbs) which have been selected based on their high selectivity and affinity for a given antigen, their long circulating half-lives, and little to no immunogenicity. Thus, mAbs as protein ligands for a carefully selected biological receptor provide an ideal delivery platform for selective targeting of pharmaceutical drugs. For example, a monoclonal antibody known to bind selectively with a specific cancer-associated antigen can be used for delivery of a chemically conjugated cytotoxic agent to the tumour, via binding, internalization, intracellular processing and finally release of active catabolite. The cytotoxic agent may be small molecule toxin, a protein toxin or other formats, like oligonucleotides. As a result, the tumour cells can be selectively eradicated, while sparing normal cells which have not been targeted by the antibody. Similarly, chemical conjugation of an antibacterial drug (antibiotic) to an antibody can be applied for treatment of bacterial infections, while conjugates of anti-inflammatory drugs are under investigation for the treatment of autoimmune diseases and for example attachment of an oligonucleotide to an antibody is a potential promising approach for the treatment of neuromuscular diseases. Hence, the concept of targeted delivery of an active pharmaceutical drug to a specific cellular location of choice is a powerful approach for the treatment of a wide range of diseases, with many beneficial aspects versus systemic delivery of the same drug.

An alternative strategy to employ monoclonal antibodies for targeted delivery of a specific protein agent is by genetic fusion of the latter protein to one (or more) of the antibody's termini, which can be the N-terminus or the C-terminus on the light chain or the heavy chain (or both). In this case, the biologically active protein of interest, e.g. a protein toxin like Pseudomonas exotoxin A (PE38) or an anti-CD3 single chain variable fragment (scFv), is genetically encoded as a fusion to the antibody, possibly but not necessarily via a peptide spacer, so the antibody is expressed as a fusion protein. The peptide spacer may contain a protease-sensitive cleavage site, or not.

In the field of ADCs, a chemical linker is typically employed to attach a pharmaceutical drug to an antibody. This linker needs to possess a number of key attributes, including the requirement to be stable in plasma after drug administration for an extended period of time. A stable linker enables localization of the ADC to the projected site or cells in the body and prevents premature release of the payload in circulation, which would indiscriminately induce undesired biological response of all kinds, thereby lowering the therapeutic index of the ADC. Upon internalization, the ADC should be processed such that the payload is effectively released so it can bind to its target.

There are two families of linkers, non-cleavable and cleavable. Non-cleavable linkers consist of a chain of atoms between the antibody and the payload, which is fully stable under physiological conditions, irrespective of which organ or biological compartment the antibody-drug conjugate resides in. As a consequence, liberation of the payload from an ADC with a non-cleavable linker relies on the complete (lysosomal) degradation of the antibody after internalization of the ADC into a cell. As a consequence of this degradation, the payload will be released, still carrying the linker, as well as a peptide fragment and/or the amino acid from the antibody the linker was originally attached to. Cleavable linkers utilize an inherent property of a cell or a cellular compartment for selective release of the payload from the ADC, which generally leaves no trace of linker after metabolic processing. For cleavable linkers, there are three commonly used mechanisms: 1) susceptibility to specific enzymes, 2) pH-sensitivity, and 3) sensitivity to redox state of a cell (or its microenvironment). The cleavable linker may also contain a self-immolative unit, for example based on a para-aminobenzyl alcohol group and derivatives thereof. A linker may also contain an additional, non-functional element, often referred to as spacer or stretcher unit, to connect the linker with a reactive group for reaction with the biomolecule.

Currently, payloads utilized in ADCs primarily include microtubule-disrupting agents [e.g. auristatins such as monomethyl auristatin E (MMAE) and monomethyl auristatin F (MMAF), maytansinoids, such as DM1 and DM4, tubulysins], DNA-damaging agents [e.g., calicheamicin, pyrrolobenzodiazepines (PBD) dimers, indolinobenzodiapines dimers, duocarmycins, anthracyclins], topoisomerase inhibitors [e.g. DXd, SN-38, exatecan and derivatives thereof, simmitecan] or RNA polymerase II inhibitors [e.g. amanitin]. Although ADCs have demonstrated clinical and preclinical activity, it has been unclear what factors determine such potency in addition to antigen expression on targeted tumour cells. For example, drug:antibody ratio (DAR), ADC-binding affinity, potency of the payload, receptor expression level, internalization rate, trafficking, multiple drug resistance (MDR) status, and other factors have all been implicated to influence the outcome of ADC treatment in vitro. In addition to the direct killing of antigen-positive tumour cells, ADCs also have the capacity to kill adjacent antigen-negative tumour cells: the so-called “bystander killing” effect, as originally reported by Sahin et al, Cancer Res. 1990, 50, 6944-6948, incorporated by reference, and for example studied Sahin et al, Cancer Res. 1990, 50, 6944-6948, incorporated by reference. Generally spoken, cytotoxic payloads that are neutral will show bystander killing whereas ionic (charged) payloads do not, as a consequence of the fact that ionic species do not readily pass a cellular membrane by passive diffusion. For example, evaluation of a range of exatecan derivatives indicated that acylation of the primary amine with hydroxyacetic acid provided a derivative (DXd) with substantially enhanced bystander killer versus various aminoacylated exatecan derivatives, as disclosed by Ogitani et al, Cancer Sci. 2016, 107, 1039-1046, incorporated by reference.

ADCs are prepared by conjugation of a linker-drug with a protein, a process known as bioconjugation. Many technologies are known for bioconjugation, as summarized in G. T. Hermanson, “Bioconjugate Techniques”, Elsevier, 3^{th} Ed Ed. 2013, incorporated by reference. Two main technologies can be recognized for the preparation of ADCs by random conjugation, either based on acylation of lysine side chain or based on alkylation of cysteine side chain. Acylation of the 8-amino group in a lysine side-chain is typically achieved by subjecting the protein to a reagent based on an activated ester or activated carbonate derivative, for example SMCC is applied for the manufacturing of Kadcyla®. Main chemistry for the alkylation of the thiol group in cysteine side-chain is based on the use of maleimide reagents, as is for example applied in the manufacturing of Adcetris®. Besides standard maleimide derivatives, a range of maleimide variants are also applied for more stable cysteine conjugation, as for example demonstrated by James Christie et al., J. Contr. Rel. 2015, 220, 660-670 and Lyon et al., Nat. Biotechnol. 2014, 32, 1059-1062, both incorporated by reference. Other approaches for cysteine alkylation involve for example nucleophilic substitution of haloacetamides (typically bromoacetamide or iodoacetamide), see for example Alley et al., Bioconj. Chem. 2008, 19, 759-765, incorporated by reference, or various approaches based on nucleophilic addition on unsaturated bonds, such as reaction with acrylate reagents, see for example Bernardim et al., Nat. Commun. 2016, 7, DOI: 10.1038/ncomms13128 and Ariyasu et al., Bioconj. Chem. 2017, 28, 897-902, both incorporated by reference, reaction with phosphonamidates, see for example Kasper et al., Angew. Chem. Int. Ed. 2019, 58, 11625-11630, incorporated by reference, reaction with allenamides, see for example Abbas et al., Angew. Chem. Int. Ed. 2014, 53,7491-7494, incorporated by reference, reaction with cyanoethynyl reagents, see for example Kolodych et al., Bioconj. Chem. 2015, 26, 197-200, incorporated by reference, reaction with vinylsulfones, see for example Gil de Montes et al., Chem. Sci. 2019, 10, 4515-4522, incorporated by reference, or reaction with vinylpyridines, see for example https://iksuda.com/science/permalink/ (accessed Jan. 7, 2020). An alternative approach to antibody conjugation without reengineering of antibody involves the reduction of interchain disulfide bridges, followed addition of a payload attached to a cysteine cross-linking reagent, such as bis-sulfone reagents, see for example Balan et al., Bioconj. Chem. 2007, 18, 61-76 and Bryant et al., Mol. Pharmaceutics 2015, 12, 1872-1879, both incorporated by reference, mono- or bis-bromomaleimides, see for example Smith et al., J. Am. Chem. Soc. 2010, 132, 1960-1965 and Schumacher et al., Org. Biomol. Chem. 2014, 37,7261-7269, both incorporated by reference, bis-maleimide reagents, see for example WO₂₀₁₄₁₁₄₂₀₇, bis(phenylthio)maleimides, see for example Schumacher et al., Org. Biomol. Chem. 2014, 37,7261-7269 and Aubrey et al., Bioconj. Chem. 2018, 29, 3516-3521, both incorporated by reference, bis-bromopyridazinediones, see for example Robinson et al., RSC Advances 2017, 7, 9073-9077, incorporated by reference, bis(halomethyl)benzenes, see for example Ramos-Tomillero et al., Bioconj. Chem. 2018, 29, 1199-1208, incorporated by reference or other bis(halomethyl)aromatics, see for example WO₂₀₁₃₁₇₃₃₉₁. Typically, ADCs prepared by cross-linking of cysteines have a drug-to-antibody loading of ˜4 (DAR4). Another useful technology for conjugation to a cysteine side chain is by means of disulfide bond, a bioactivatable connection that has been utilized for reversibly connecting protein toxins, chemotherapeutic drugs, and probes to carrier molecules (see for example Pillow et al., Chem. Sci. 2017, 8, 366-370, incorporated by reference).

Besides conjugation to lysine or cysteine, a range of other conjugation technologies has been explored in the past decade. One method is based on genetic encoding of a non-natural amino acid, e.g. p-acetophenylalanine suitable for oxime ligation, or p-azidomethylphenylalanine or p-azidophenylalanine suitable for click chemistry conjugation, as for example demonstrated by Axup et al. Proc. Nat. Acad. Sci. 2012, 109, 16101-16106, incorporated by reference. Similarly,

Zimmerman et al., Bioconj. Chem. 2014, 25, 351-361, incorporated by reference have employed a cell-free protein synthesis method to introduce azidomethylphenylalanine (AzPhe) into monoclonal antibodies for conversion into ADC by means of metal-free click chemistry. Also, it has also be shown by Nairn et al., Bioconj. Chem. 2012, 23, 2087-2097, incorporated by reference, that a methionine analogue like azidohomoalanine (Aha) can be introduced into protein by means of auxotrophic bacteria and further converted into protein conjugates by means of (copper-catalysed) click chemistry. Finally, genetic encoding of aliphatic azides in recombinant proteins using a pyrrolysyl-tRNA synthetase/tRNAcuA pair was shown by Nguyen et al., J. Am. Chem. Soc. 2009, 131, 8720-8721, incorporated by reference and labelling was secured by click chemistry.

Another method is based on enzymatic installation of a non-natural functionality. For example, Lhospice et al., Mol. Pharmaceut 2015, 12, 1863-1871, incorporated by reference, employ the bacterial enzyme transglutaminase (BTG or TGase) for installation of an azide moiety onto an antibody. A genetic method based on C-terminal TGase-mediated azide introduction followed by conversion in ADC with metal-free click chemistry was reported by Cheng et al., Mol. Cancer Therap. 2018, 17, 2665-2675, incorporated by reference.

It has been shown in WO₂₀₁₄₀₆₅₆₆₁, by van Geel et al., Bioconj. Chem. 2015, 26, 2233-2242 and Verkade et al., Antibodies 2018, 7, 12, all incorporated by reference, that enzymatic remodelling of the native antibody glycan at N297 enables introduction of an azido-modified sugar, suitable for attachment of cytotoxic payload using click chemistry.

Chemical approaches have also been developed for site-specific modification of antibodies without prior genetic modification, as for example highlighted by Yamada and Ito, ChemBioChem. 2019, 20, 2729-2737.

Besides reaction between azides and cyclooctynes, bioconjugation of linker-drugs to proteins (and other biomolecules such as glycans, nucleic acids) can be achieved by a range of other metal-free click chemistries as well, see e.g. Nguyen and Prescher, Nature rev. 2020, doi:

10.1038/s41570-020-0205-0, incorporated by reference. For example, oxidation of a specific tyrosine in a protein can give an ortho-quinone , which readily undergoes cycloaddition with strained alkenes (e.g. TCO) or strained alkynes, see e.g. Bruins et al., Chem. Eur. J. 2017, 24, 4749-4756, incorporated by reference. Besides cyclooctyne, certain cycloheptynes are also suitable for metal-free click chemistry, as reported by Wetering et al. Chem. Sci. 2020, doi: 10.1039/d0sc03477k, incorporated by reference. A tetrazine moiety can also be introduced into a protein or a glycan by various means, for example by genetic encoding or chemical acylation, and may also undergo cycloaddition with cyclic alkenes and alkynes. A list of couples of functional groups F and Q for metal-free click chemistry is provided in FIG. 1.

Based on the above, a general method for the preparation of a protein conjugate, exemplified for a monoclonal antibody in FIG. 2, entails the reaction of a protein containing x number of reactive moieties F with a linker-drug construct containing a single molecule Q. A schematic depiction how reactive molecules F can be introduced into a monoclonal antibody is provided FIG. 3.

A frequent method for bioconjugation of linker-drugs to azido-modified proteins is strain-promoted alkyne-azide cycloaddition (SPAAC). In a SPAAC reaction, the linker-drug is functionalized with a cyclic alkyne and the cycloaddition is driven by relief of ring-strain. Various strained alkynes suitable for metal-free click chemistry, as for example with azide, quinones or nitrile oxides, are indicated in FIG. 4.

Conjugation of a cytotoxic payload to an antibody by any of the methods described above is often challenging due to the hydrophobic nature of the payload and in some cases in combination with the linker, which encumbers solubility in aqueous or buffered systems (the preferred medium for antibodies). As a consequence, conjugation of cytotoxic payload is typically performed in medium consisting of water/buffer plus an organic co-solvent. Typical co-solvents for conjugation are DMSO, propylene glycol (PG), ethanol, DMF, DMA and NMP, which facilitate solubilization of linker-drug but can also mix well with water. Typical amount of co-solvent is 10-25% versus aqueous medium, however, co-solvents may be added up to 50% in some cases. Adding high amount of co-solvent is particularly favourable for conjugation processes where the payload is significantly hydrophobic (lipophilic) and in those processes where a large excess of linker-drug is required to achieve full conversion to desired product.

Besides the apparent benefits, the downside of adding a significant amount of organic co-solvent is that the antibody may not be stable in the solvent mixture and as a consequence may aggregate during the conjugation process. Typically, aggregation levels will be correlated to the amount of co-solvent, but this is also antibody-dependent. Especially for unstable antibodies, aggregation levels may be significant, reaching levels of 10% or even more, which will consequently compromise process yields. Moreover, these levels of aggregates will require an additional processing step (e.g. SEC or CHT) to remove aggregates to an acceptable level.

An additional disadvantage of high co-solvent levels during conjugation is the necessity to introduce an additional process step to remove the excess co-solvent, e.g. by dialysis, by spin-filtration or by TFF, before size-exclusion purification can be performed (SEC).

Surfactants are well-known in the art. Surfactants are a general class of organic compounds composed of one polar (or ionic) hydrophilic end group attached to a non-polar hydrophobic hydrocarbon fragment. This amphiphilic property contributes to the unique phase behaviour that lowers the surface tension between two phases. Surfactants are widely applied throughout various industries, including detergent, paints, plastics, cosmetics, agriculture, and pharmaceuticals. In the pharmaceutical industry, surfactants have been mainly used in formulations to improve drug solubility and stability in liquid form, for viral and bacterial inactivation, in upstream bioprocessing to enhance protein secretion and in downstream bioprocesses to separate proteins from cells and tissues.

It has been reported by Hu et al, Bioconj. Chem. 2018, 29, 3667-3676, incorporated by reference, that the surfactant sodium decanoate is used in the drug substance process of Besponsa, an antibody drug conjugate (ADC), to facilitate bioconjugation via lysine acylation chemistry between activated calicheamicin derivative (linker payload) and inotuzumab (monoclonal antibody). The micelle formation was shown to be critical for the efficient conjugation reaction. Further screening studies indicated that sodium dodecyl sulfate, sodium deoxycholate, and dodecyltrimethylammonium bromide were also able to facilitate the conjugation reaction. It was also shown, however, that the charge of surfactant and the choice of linker payload influence the conjugated lysine site selectivity. Eight major conjugated lysine sites are observed in Besponsa, as compared to approximately 80 conjugated lysine sites typically observed for conjugation of lysine in antibody without surfactants.

Anionic surfactants have also been shown to enhance protein conjugation processes using click chemistry. Specifically, has been reported by Schneider et al., Bioorg. Med. Chem. 2016, 24, 995-1001, incorporated by reference, that anionic surfactants enhance conjugate formation using copper-catalyzed click chemistry (CuAAC) by up to 10-fold resulting in high yields even at low (i.e., micromolar) concentrations of the reactants. However, it was not shown whether protein conjugation based on strain-promoted (metal-free) click chemistry (SPAAC) is also improved. One study by Anderton et al., Bioconj. Chem. 2015, 26, 1687-1691, incorporated by reference, mentions that strain-promoted azide—alkyne cycloaddition (SPAAC) can also be improved by using micellar catalysis with anionic and cationic surfactants, with rate enhancements of up to 179-fold for reaction of benzyl azide with DIBAC cyclooctyne. A more modest 11- fold rate enhancement is observed for micellar catalysis of the reaction between benzyl azide and a DIBAC-functionalized DNA sequence, demonstrating that micellar catalysis can be successfully applied to nucleic acids.

### SUMMARY OF THE INVENTION

The present inventors have surprisingly found a bioconjugation reaction between a biomolecule that is functionalized with a click probe F and an alkyne- or alkene-functionalized payload can be greatly improved by the addition of a surfactant to the reaction mixture. In the presence of the surfactant, higher conversions (and thus higher yields and drug-to-antibody ratios closer to the theoretical value) were obtained. Furthermore, the reaction could be performed with less organic co-solvent and a higher concentration of biomolecule, which in turn led to less aggregation of the conjugated biomolecule and easier purification. Also, the conjugation reaction performed well with a lower excess of alkyne- or alkene-functionalized payload, thus requiring less of an expensive and synthetically complex molecule in the preparation of bioconjugates.

The invention can be defined by the following list of preferred embodiments:

1. A method for the preparation of a bioconjugate of structure B—(Z—L—D)ₓ (1), comprising reacting:
(i) an alkyne or alkene compound of structure Q—L—D (2), wherein
   Q is a click probe comprising a cyclic alkyne moiety or a cyclic alkene moiety,
   L is a linker, and
   D is a payload;

with
(ii) a molecule of structure B—(F)ₓ (3), wherein
   B is a biomolecule that is functionalized with x click probes F;
   F is a click probe capable of reacting with Q, and
   x is an integer in the range of 1-10,

in presence of a surfactant, to form a bioconjugate wherein the payload is covalently attached to the biomolecule via connecting group Z that is formed by a click reaction between Q and F.

2. The method according to embodiment 1, wherein the surfactant contains a negatively charged moiety, preferably wherein the surfactant is anionic.

3. The method according to embodiment 1 or 2, wherein the surfactant is selected from the group consisting of sodium decanoate, sodium dodecanoate, sodium lauryl sulfate (SDS), sodium deoxycholate, preferably wherein the surfactant is sodium decanoate or sodium deoxycholate.

4. The method according to any one of the preceding embodiments, wherein the reaction is performed in a solvent system containing water and organic solvent in a ratio in the range of 50/50-100/0, preferably in the range of 75/25-95/5.

5. The method according to any one of the preceding embodiments, wherein the concentration of the molecule of structure (3) is in the range of 1-100 mg/mL, preferably in the range 5-50 mg/mL, more preferably in the range of 10-20 mg/mL.

6. The method according to any one of the preceding embodiments, wherein the click probe Q comprises a cyclic alkyne moiety and click probe F is selected from the group consisting of azide, tetrazine, triazine, nitrone, nitrile oxide, nitrile imine, diazo compound, ortho-quinone, dioxothiophene and sydnone, preferably click probe F is an azide moiety.

7. The method according to any one of the preceding embodiments, wherein the click probe Q is selected from the group consisting of (Q22)-(Q36):

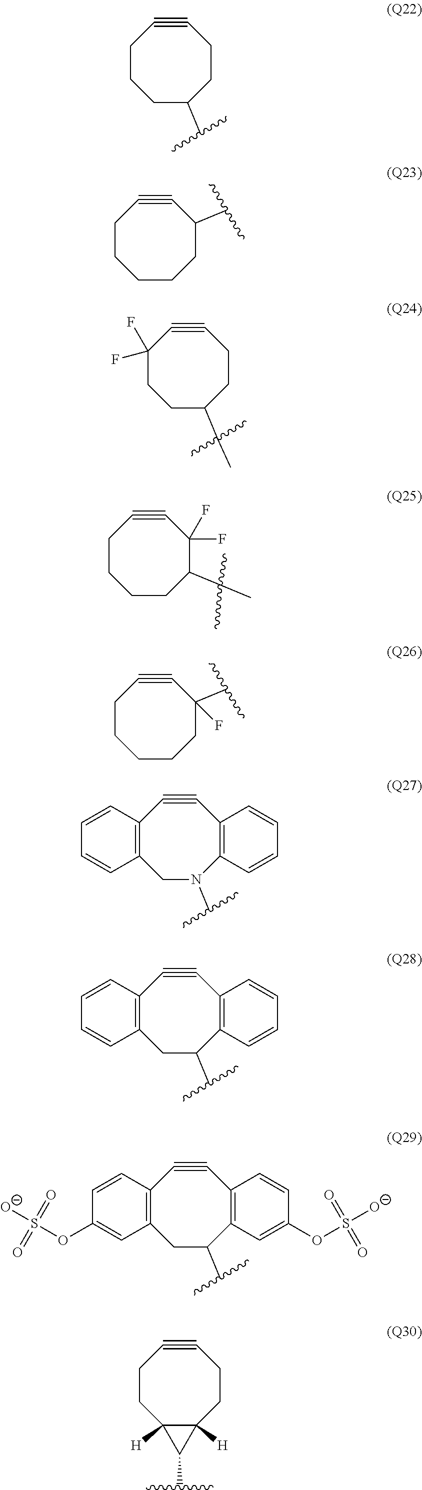

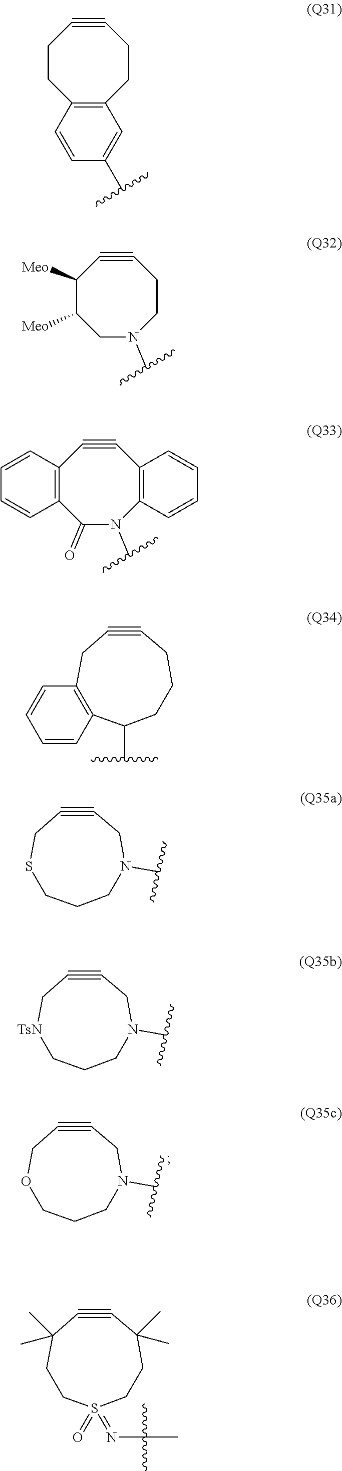

or wherein the (hetero)cycloalkynyl moiety Q is according to structure (Q37):

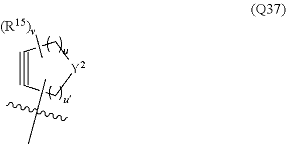

wherein:

R¹⁵ is independently selected from the group consisting of hydrogen, halogen, —OR¹⁶, —NO₂, —CN, —S(O)₂R¹⁶, —S(O)₃^{(-) , C}₁-C₂₄ alkyl groups, C₆-C₂₄ (hetero)aryl groups, C₇-C₂₄ alkyl(hetero)aryl groups and C₇-C₂₄ (hetero)arylalkyl groups and wherein the alkyl groups, (hetero)aryl groups, alkyl(hetero)aryl groups and (hetero)arylalkyl groups are optionally substituted, wherein two substituents R¹⁵ may be linked together to form an optionally substituted annulated cycloalkyl or an optionally substituted annulated (hetero)arene substituent, and wherein R¹⁶ is independently selected from the group consisting of hydrogen, halogen, C₁-C₂₄ alkyl groups, C₆-C₂₄ (hetero)aryl groups, C₇-C₂₄ alkyl(hetero)aryl groups and C₇-C₂₄ (hetero)arylalkyl groups;

y² is C(R³¹)₂, O, S or NR³¹, wherein each R³¹ individually is R¹⁵ or —LD;

u is 0, 1, 2, 3, 4 or 5;

u′ is 0, 1, 2, 3, 4 or 5, wherein u+u′=4, 5, 6, 7 or 8;

v=an integer in the range 8-16;

preferably wherein the cyclooctynyl moiety Q is according to structure (Q38):

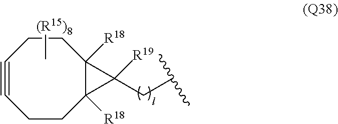

wherein
R¹⁵ is independently selected from the group consisting of hydrogen, halogen, —OR¹⁶, —NO₂, —CN, —S(O)₂R¹⁶, —S(O)₃⁽⁻⁾, C₁-C₂₄ alkyl groups, C₅-C₂₄ (hetero)aryl groups, C₇-C₂₄ alkyl(hetero)aryl groups and C₇-C₂₄ (hetero)arylalkyl groups and wherein the alkyl groups, (hetero)aryl groups, alkyl(hetero)aryl groups and (hetero)arylalkyl groups are optionally substituted, wherein two substituents R¹⁵ may be linked together to form an optionally substituted annulated cycloalkyl or an optionally substituted annulated (hetero)arene substituent, and wherein R¹⁶ is independently selected from the group consisting of hydrogen, halogen, C₁-C₂₄ alkyl groups, C₆-C₂₄ (hetero)aryl groups, C₇-C₂₄ alkyl(hetero)aryl groups and C₇-C₂₄ (hetero)arylalkyl groups;
R¹⁸ is independently selected from the group consisting of hydrogen, halogen, C₁-C₂₄ alkyl groups, C₆-C₂₄ (hetero)aryl groups, C₇-C₂₄ alkyl(hetero)aryl groups and C₇-C₂₄ (hetero)arylalkyl groups;

R¹⁹ is selected from the group consisting of hydrogen, —LD; halogen, C₁-C₂₄ alkyl groups, C₆-C₂₄ (hetero)aryl groups, C₇-C₂₄ alkyl(hetero)aryl groups and C₇-C₂₄ (hetero)arylalkyl groups, the alkyl groups optionally being interrupted by one of more hetero-atoms selected from the group consisting of O, N and S, wherein the alkyl groups, (hetero)aryl groups, alkyl(hetero)aryl groups and (hetero)arylalkyl groups are independently optionally substituted; and
I is an integer in the range 0 to 10;

or wherein the (hetero)cyclooctynyl moiety Q is according to structure (Q39):

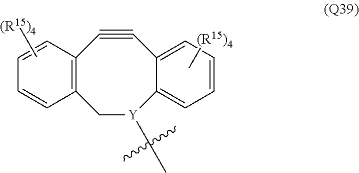

wherein
R¹⁵ is independently selected from the group consisting of hydrogen, halogen, —OR¹⁶, —NO₂, —CN, —S(O)₂R¹⁶, —S(O)₃⁽⁻⁾, C₁-C₂₄ alkyl groups, Cs-C₂₄ (hetero)aryl groups, C₇-C₂₄ alkyl(hetero)aryl groups and C₇-C₂₄ (hetero)arylalkyl groups and wherein the alkyl groups, (hetero)aryl groups, alkyl(hetero)aryl groups and (hetero)arylalkyl groups are optionally substituted, wherein two substituents R¹⁵ may be linked together to form an optionally substituted annulated cycloalkyl or an optionally substituted annulated (hetero)arene substituent, and wherein R¹⁶ is independently selected from the group consisting of hydrogen, halogen, C₁-C₂₄ alkyl groups, C₆-C₂₄ (hetero)aryl groups, C₇-C₂₄ alkyl(hetero)aryl groups and C₇-C₂₄ (hetero)arylalkyl groups;
Y is N or CR¹⁵.

8. The method according to any one of embodiments 1-6, wherein the click probe Q is selected from the group consisting of, optionally substituted, (hetero)cyclopropenyl group, (hetero)cyclobutenyl group, trans-(hetero)cycloheptenyl group, trans-(hetero)cyclooctenyl group, trans-(hetero)cyclononenyl group or trans-(hetero)cyclodecynyl group, preferably click probe Q is selected from the group consisting of (Q40)-(Q50):

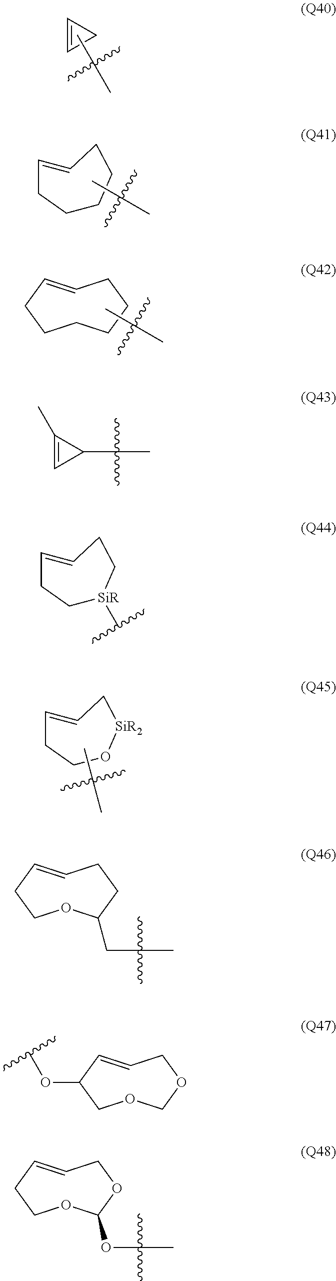

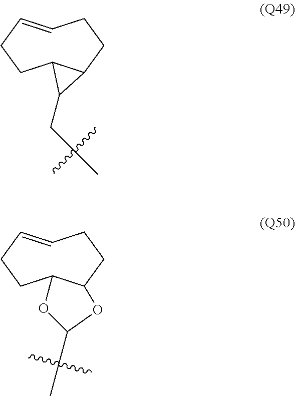 [SMILES: *CC1C2CC/C=C/CCC12 ; *C1OC2CC/C=C/CCC2O1]

wherein the R group(s) on Si in (Q44) and (Q45) is alkyl or aryl.

9. The method according to any one of the preceding embodiments, wherein the payload D is a cytotoxin, preferably a cytotoxin selected from colchicine, vinca alkaloids, anthracyclines, camptothecins, doxorubicin, daunorubicin, taxanes, calicheamycins, tubulysins, irinotecans, an inhibitory peptide, amanitin, deBouganin, duocarmycins, maytansines, auristatins, enediynes, pyrrolobenzodiazepines (PBDs) or indolinobenzodiazepine dimers (IGN) or PNU-159,682 and derivatives thereof, more preferably calicheamicin, PBD dimer, SN-38, MMAE or exatecan.

10. The method according to any one of the preceding embodiments, wherein the biomolecule is selected from the group consisting of proteins (including glycoproteins such as antibodies), polypeptides, peptides, glycans, lipids, nucleic acids, oligonucleotides, polysaccharides, oligosaccharides, enzymes, hormones, amino acids and monosaccharides, more preferably from the group consisting of proteins, polypeptides, peptides and glycans, most preferably the biomolecule is a protein.

11. The method according to embodiment 10, wherein the biomolecule is selected from the group consisting of mAb, Fab, VHH, scFv, diabody, minibody, affibody, affylin, affimers, atrimers, fynomer, Cys-knot, DARPin, adnectin/centryin, knottin, anticalin, FN3, Kunitz domain, OBody, bicyclic peptides and tricyclic peptides.

12. The method according to any one of the preceding embodiments, wherein the click probe F is connected to a monosaccharide moiety, preferably to the terminal monosaccharide moiety of a glycan of a glycoprotein, most preferably of an antibody.

13. Use of a surfactant in bioconjugation reaction to prepare a bioconjugate of structure B—(Z—L—D)ₓ (1), wherein x payloads D are covalently attached to a biomolecule B via connecting group Z that is formed by a click reaction of click probe Q with click probe F, wherein the reaction is between:
(i) an alkyne or alkene compound of structure Q—L—D (2), wherein
   Q is a click probe comprising a cyclic alkyne moiety or a cyclic alkene moiety,
   L is a linker, and
   D is a payload;

with
(ii) a molecule of structure B—(F), (3), wherein
   B is a biomolecule that is functionalized with x click probes F;
   F is a click probe capable of reacting with Q, and
   x is an integer in the range of 1-10.

14. The use according to embodiment 13, which is for one or more of:
(i) increasing the conversion of the bioconjugation reaction;
(ii) increasing the yield of the bioconjugation reaction;
(iii) reducing the amount of organic co-solvent in the solvent system wherein the bioconjugation reaction is performed;
(iv) providing flexibility in the concentration of biomolecule during the bioconjugation reaction;
(v) reducing the excess of alkyne- or alkene-functionalized payload used during the bioconjugation reaction;
(vi) reducing the extent of aggregate formation during the bioconjugation reaction;
(vii) simplifying downstream processing of the bioconjugate.

15. The use according to embodiment 13 or 14, for improving the drug-to-antibody ratio (DAR) of the bioconjugate.

### DESCRIPTION OF THE FIGURES

FIG. 1 shows a representative (but not comprehensive) set of functional groups (F) that can be introduced into a biomolecule by engineering, by chemical modification, or by enzymatic means, which upon metal-free click reaction with a complementary reactive group Q lead to connecting group Z. Functional group F may be artificially introduced (engineered) into a biomolecule at any position of choice. Some functional groups F (e.g. nitrile oxide, quinone), may besides strained alkynes also react with strained alkenes, which as an example is depicted for triazine or tetrazine (bottom line). The pyridine or pyridazine connecting group is the product of the rearrangement of the tetrazabicyclo[2.2.2]octane connecting group, formed upon reaction of triazine or tetrazine with alkyne (but not alkene), respectively, with loss of Nz. Connecting groups Z are preferred connecting groups to be used in the present invention.

FIG. 2 shows the general scheme for preparation of antibody-drug conjugates by reaction of a monoclonal antibody (in most cases a symmetrical dimer) containing an x number of functionalities F. By incubation of antibody-(F)ₓ with excess of a linker-drug construct (Q-spacer-linker-payload) a conjugate is obtained by reaction of F with Q, forming bond Z.

FIG. 3 shows the general process for non-genetic conversion of a monoclonal antibody into an antibody containing probes for click conjugation (F). The click probe may be on various positions in the antibody, depending on the technology employed. For example, the antibody may be converted into an antibody containing two click probes (structure on the left) or four click probes (bottom structure) or eight probes (structure on the right) for click conjugation.

FIG. 4 shows cyclic alkynes suitable for metal-free click chemistry, and preferred embodiments for reactive moiety Q. The list is not comprehensive, for example alkynes can be further activated by fluorination, by substitution of the aromatic rings or by introduction of heteroatoms in the aromatic ring.

FIG. 5 depicts a specific example of site-specific conjugation of a payload based on glycan remodeling of a full-length IgG followed by azide-cyclooctyne click chemistry. The IgG is first enzymatically remodeled by endoglycosidase-mediated trimming of all different glycoforms, followed by glycosyltransferase-mediated transfer of azido-sugar onto the core GlcNAc liberated by endoglycosidase. In the next step, the azido-remodeled IgG is subjected to a polypeptide, which has been modified with a single cyclooctyne for metal-free click chemistry (SPAAC), leading to a bispecific antibody of 2:2 molecular format. It is also depicted that the cyclooctyne-polypeptide construct will have a specific spacer between cyclooctyne and polypeptide, which enables tailoring of IgG-polypeptide distance or impart other properties onto the resulting bispecific antibody.

FIG. 6 show a plot demonstrating the impact of various surfactants on the conjugation efficiency of linker-drugs X1 and X2 to azido-remodeled antibodies obtained by the process depicted in FIG. 5. X1 or X2 react with the cyclooctyne part with the azido-remodeled antibodies as a result of metal-free click chemistry. Clearly, the addition of surfactants leads to an improved drug-to-antibody ratio (DAR).

FIGS. 7A-7F show the structures of BCN-containing linker-drugs X1 to X12.

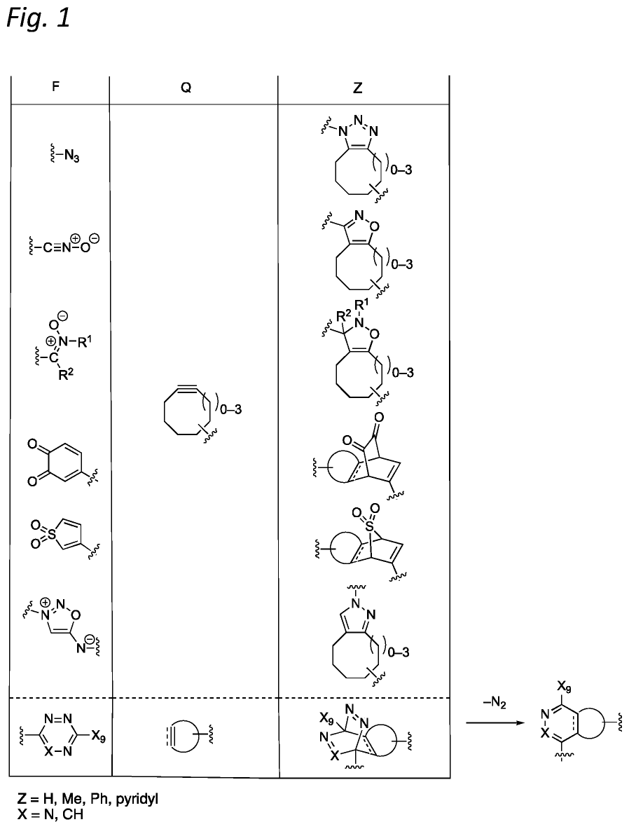

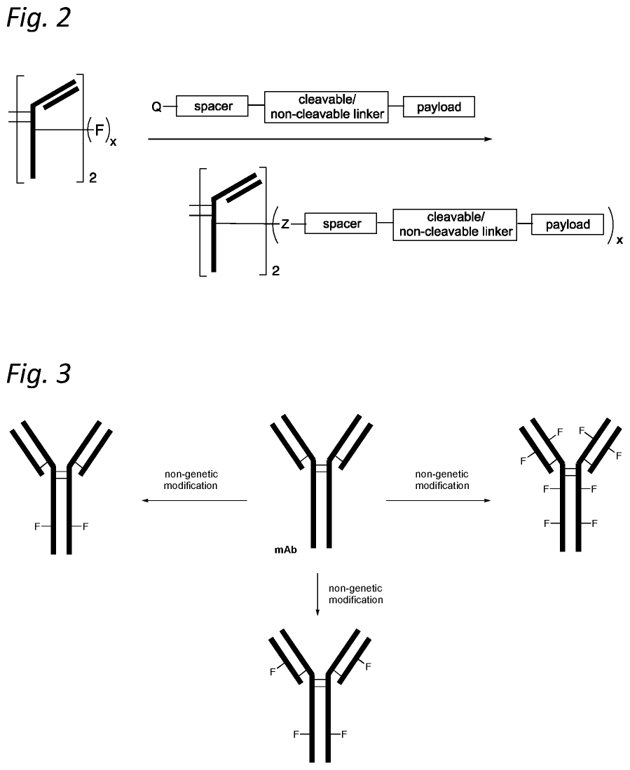

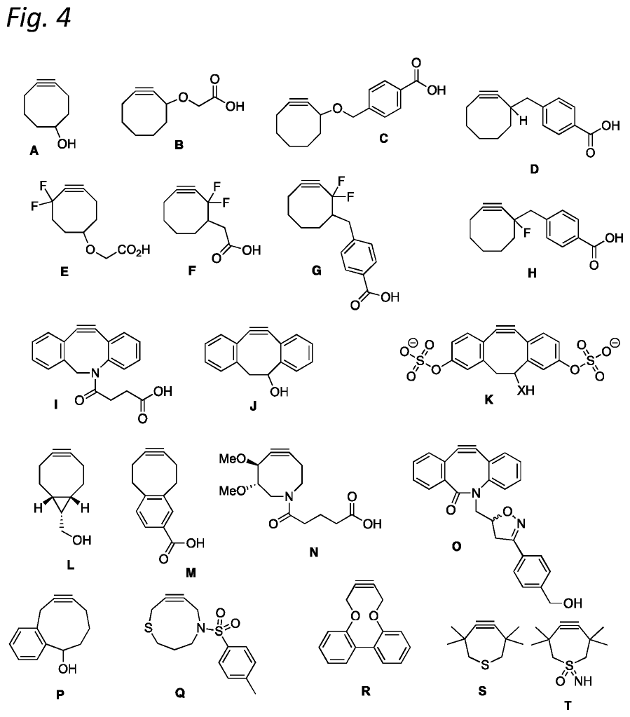

### DETAILED DESCRIPTION OF THE INVENTION

### Definitions

The verb “to comprise”, and its conjugations, as used in this description and in the claims is used in its non-limiting sense to mean that items following the word are included, but items not specifically mentioned are not excluded. In addition, reference to an element by the indefinite article “a” or “an” does not exclude the possibility that more than one of the element is present, unless the context clearly requires that there is one and only one of the elements. The indefinite article “a” or “an” thus usually means “at least one”.

The compounds disclosed in this description and in the claims may comprise one or more asymmetric centres, and different diastereomers and/or enantiomers may exist of the compounds. The description of any compound in this description and in the claims is meant to include all diastereomers, and mixtures thereof, unless stated otherwise. In addition, the description of any compound in this description and in the claims is meant to include both the individual enantiomers, as well as any mixture, racemic or otherwise, of the enantiomers, unless stated otherwise. When the structure of a compound is depicted as a specific enantiomer, it is to be understood that the invention of the present application is not limited to that specific enantiomer.

The compounds may occur in different tautomeric forms. The compounds according to the invention are meant to include all tautomeric forms, unless stated otherwise. When the structure of a compound is depicted as a specific tautomer, it is to be understood that the invention of the present application is not limited to that specific tautomer.

The compounds disclosed in this description and in the claims may further exist as exo and endo diastereoisomers. Unless stated otherwise, the description of any compound in the description and in the claims is meant to include both the individual exo and the individual endo diastereoisomers of a compound, as well as mixtures thereof. When the structure of a compound is depicted as a specific endo or exo diastereomer, it is to be understood that the invention of the present application is not limited to that specific endo or exo diastereomer.

The compounds according to the invention may exist in salt form, which are also covered by the present invention. The salt is typically a pharmaceutically acceptable salt, containing a pharmaceutically acceptable anion. The term “salt thereof” means a compound formed when an acidic proton, typically a proton of an acid, is replaced by a cation, such as a metal cation or an organic cation and the like. Where applicable, the salt is a pharmaceutically acceptable salt, although this is not required for salts that are not intended for administration to a patient. For example, in a salt of a compound the compound may be protonated by an inorganic or organic acid to form a cation, with the conjugate base of the inorganic or organic acid as the anionic component of the salt.

The term “pharmaceutically accepted” salt means a salt that is acceptable for administration to a patient, such as a mammal (salts with counter ions having acceptable mammalian safety for a given dosage regime). Such salts may be derived from pharmaceutically acceptable inorganic or organic bases and from pharmaceutically acceptable inorganic or organic acids. “Pharmaceutically acceptable salt” refers to pharmaceutically acceptable salts of a compound, which salts are derived from a variety of organic and inorganic counter ions known in the art and include, for example, sodium, potassium, calcium, magnesium, ammonium, tetraalkylammonium, etc., and when the molecule contains a basic functionality, salts of organic or inorganic acids, such as hydrochloride, hydrobromide, formate, tartrate, besylate, mesylate, acetate, maleate, oxalate, etc.

The term “protein” is herein used in its normal scientific meaning. Herein, polypeptides comprising about 10 or more amino acids are considered proteins. A protein may comprise natural, but also unnatural amino acids.

The term “antibody” is herein used in its normal scientific meaning. An antibody is a protein generated by the immune system that is capable of recognizing and binding to a specific antigen. An antibody is an example of a glycoprotein. The term antibody herein is used in its broadest sense and specifically includes monoclonal antibodies, polyclonal antibodies, dimers, multimers, multi-specific antibodies (e.g. bispecific antibodies), antibody fragments, and double and single chain antibodies. The term “antibody” is herein also meant to include human antibodies, humanized antibodies, chimeric antibodies and antibodies specifically binding cancer antigen. The term “antibody” is meant to include whole immunoglobulins, but also antigen-binding fragments of an antibody. Furthermore, the term includes genetically engineered antibodies and derivatives of an antibody. Antibodies, fragments of antibodies and genetically engineered antibodies may be obtained by methods that are known in the art. Typical examples of antibodies include, amongst others, abciximab, rituximab, basiliximab, palivizumab, infliximab, trastuzumab, efalizumab, alemtuzumab, adalimumab, tositumomab, cetuximab, ibrituximab, omalizumab, bevacizumab, natalizumab, ranibizumab, panitumumab, eculizumab, certolizumab pegol, golimumab, canakinumab, catumaxomab, ustekinumab, tocilizumab, ofatumumab, denosumab, belimumab, ipilimumab and brentuximab.

An “antibody fragment” is herein defined as a portion of an intact antibody, comprising the antigen-binding or variable region thereof. Examples of antibody fragments include Fab, Fab′, F(ab′)₂, and Fv fragments, diabodies, minibodies, triabodies, tetrabodies, linear antibodies, single-chain antibody molecules, scFv, scFv-Fc, multispecific antibody fragments formed from antibody fragment(s), a fragment(s) produced by a Fab expression library, or an epitope-binding fragments of any of the above which immunospecifically bind to a target antigen (e.g., a cancer cell antigen, a viral antigen or a microbial antigen).

An “antigen” is herein defined as an entity to which an antibody specifically binds.

The terms “specific binding” and “specifically binds” is herein defined as the highly selective manner in which an antibody or antibody binds with its corresponding epitope of a target antigen and not with the multitude of other antigens. Typically, the antibody or antibody derivative binds with an affinity of at least about 1x10⁻⁷ M, and preferably 10⁻⁸ M to 10⁻⁹ M, 10⁻¹⁰ M, 10⁻¹¹ M, or 10⁻¹² M and binds to the predetermined antigen with an affinity that is at least two-fold greater than its affinity for binding to a non-specific antigen (e.g., BSA, casein) other than the predetermined antigen or a closely-related antigen.

The term “substantial” or “substantially” is herein defined as a majority, i.e. >50% of a population, of a mixture ora sample, preferably more than 50%, 55%, 60%, 65%, 70%, 75%, 80%, 85%, 90%, 91%, 92%, 93%, 94%, 95%, 96%, 97%, 98%, or 99% of a population.

A “linker” is herein defined as a moiety that connects two or more elements of a compound. For example in an antibody-conjugate, an antibody and a payload are covalently connected to each other via a linker. A linker may comprise one or more linkers and spacer-moieties that connect various moieties within the linker.

A “spacer” or spacer-moiety is herein defined as a moiety that spaces (i.e. provides distance between) and covalently links together two (or more) parts of a linker. The linker may be part of e.g. a linker-construct, the linker-conjugate or a bioconjugate, as defined below.

A “self-immolative group” is herein defined as a part of a linker in an antibody-drug conjugate with a function is to conditionally release free drug at the site targeted by the ligand unit. The activatable self-immolative moiety comprises an activatable group (AG) and a self-immolative spacer unit. Upon activation of the activatable group, for example by enzymatic conversion of an amide group to an amino group or by reduction of a disulfide to a free thiol group, a self-immolative reaction sequence is initiated that leads to release of free drug by one or more of various mechanisms, which may involve (temporary) 1,6-elimination of a p-aminobenzyl group to a p-quinone methide, optionally with release of carbon dioxide and/or followed by a second cyclization release mechanism. The self-immolative assembly unit can part of the chemical spacer connecting the antibody and the payload (via the functional group). Alternatively, the self-immolative group is not an inherent part of the chemical spacer, but branches off from the chemical spacer connecting the antibody and the payload.

A “bioconjugate” is herein defined as a compound wherein a biomolecule is covalently connected to a payload via a linker. A bioconjugate comprises one or more biomolecules and/or one or more payloads. Antibody-conjugates, such as antibody-payload conjugates and antibody-drug-conjugates are bioconjugates wherein the biomolecule is an antibody.

A “biomolecule” is herein defined as any molecule that can be isolated from nature or any molecule composed of smaller molecular building blocks that are the constituents of macromolecular structures derived from nature, in particular nucleic acids, proteins, glycans and lipids. Examples of a biomolecule include an enzyme, a (non-catalytic) protein, a polypeptide, a peptide, an amino acid, an oligonucleotide, a monosaccharide, an oligosaccharide, a polysaccharide, a glycan, a lipid and a hormone.

The term “payload” refers to the moiety that is covalently attached to a targeting moiety such as an antibody, but also to the molecule that is released from the conjugate upon uptake of the protein conjugate and/or cleavage of the linker. Payload thus refers to the monovalent moiety having one open end which is covalently attached to the targeting moiety via a linker, which is in the context of the present invention referred to as D, and also to the molecule that is released therefrom.

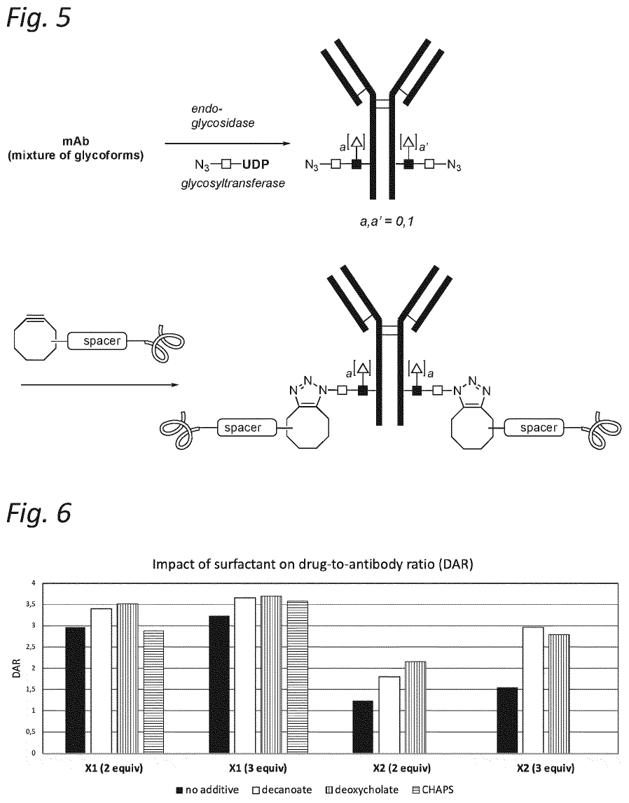

The term “drug-to-antibody ratio” or “DAR” refers to the number of payloads that are connected to a biomolecule. In the context of the term DAR, “drug” should not be narrowly construed but refers to any payload suitable in the context of the present invention, although preferably the payload is a drug. Likewise, in the context of the term DAR, “antibody” should not be narrowly construed but refers to any biomolecule suitable in the context of the present invention, although preferably the biomolecule is an antibody. The term DAR may thus also be referred to as “payload-to-biomolecule ratio”. Any bioconjugation reaction and obtained bioconjugate has a theoretical DAR, determined by the amount of conjugation sites on the biomolecule and payload moieties that are attached per conjugation site. For example, the conjugation reaction depicted in FIG. 5 involves an antibody with two conjugation sites (azido moieties) and the attachment of one payload (polypeptide) per conjugation site, leading to a theoretical DAR of 2. In practice, bioconjugation reactions may have a conversion below 100 )/0, leading to a DAR for the obtained conjugate that is lower than the theoretical DAR.

The term “surfactant” or surface-active agent refers to a class of compounds that lower the surface tension between two liquids. Surfactants are amphiphilic organic molecules that contain both a hydrophobic group (usually referred to as their “tail”) and a hydrophilic group (usually referred to as their “head”). Surfactants may be non-ionic, anionic, cationic and zwitterionic. In case the surfactant is part of a salt, such as for example sodium dodecyl sulfate (SDS), only the amphiphilic ion is referred to as the surfactant, here the dodecyl sulfate anion, wherein the dodecyl part is lipophilic and the sulfate is hydrophilic. The presence of cationic sodium is further irrelevant for the surfactant nature of SDS, which is thus an anionic surfactant.

The invention

The inventors have surprisingly found a bioconjugation reaction between a biomolecule that is functionalized with a click probe F that is able to react with payload functionalized with a cyclic alkyne or alkene moiety Q, which can be greatly improved by the addition of a surfactant to the reaction mixture. In the presence of a surfactant, higher conversions (and thus yields) were obtained. Furthermore, the reaction could be performed with less organic co-solvent and a higher concentration of biomolecule, which in turn led to easier purification. Also, the conjugation reaction performed well with a lower excess of cyclic alkyne- or alkene-functionalized payload, thus requiring less of an expensive and synthetically complex molecule in the preparation of bioconjugates. The present invention thus resides in a bioconjugation reaction in the presence of a surfactant and in the use of a surfactant in bioconjugation.

The use of surfactants to improve the bioconjugation reaction between a cyclic alkyne or alkene compound and a hydrophilic azide moiety, as subject of the present invention, is unprecedented in the art, especially in the context of strain-promoted alkyne-azide ligation. The rare use of surfactants to enhance metal-free click reaction involves hydrophobic azide moieties, the solubility of which is improved using surfactants. In the present invention, the solubility of the azide moiety is of no concern. Yet, the inventors have found a marked improvement in efficiency of the conjugation reaction for azides that are already highly soluble in aqueous system.

More specifically, the invention provides in a first aspect a method for the preparation of a bioconjugate of structure B—(Z—L—D)ₓ (1), comprising reacting (i) a cyclic alkyne or a cyclic alkene compound of structure Q—L—D (2), wherein Q is a cyclic alkyne or a cyclic alkene moiety, L is a linker and D is a payload, with (ii) a molecule of structure B—(F)ₓ (3), wherein B is a biomolecule that is functionalized with x click probes F, and x is an integer in the range of 1-10, in presence of a surfactant, to form a bioconjugate wherein the payload is covalently attached to the biomolecule via connecting group Z that is formed by a click reaction, typically a 1,3-dipolar cycloaddition or (4+2) cycloaddition,of click probe Q with click probe F.

The invention in a second aspect provides the use of a surfactant in a bioconjugation reaction to prepare a bioconjugate of structure B—(Z—L—D)ₓ (1), wherein x payloads D are covalently attached to a biomolecule B via connecting group Z which contains a moiety that is formed by 1,3-dipolar cycloaddition or a (4+2)-cycloaddition of a cyclic alkyne or a cyclic alkene moiety with click probe F, wherein the reaction is between (i) an alkyne compound of structure Q—L—D (2), wherein Q is a cyclic alkyne or a cyclic alkene moiety, L is a linker and D is a payload, and (ii) a molecule of structure B—(F)ₓ (3), wherein B is a biomolecule that is functionalized with x click probes F, and x is an integer in the range of 1-10.

As will be clear from the context of the present invention, everything defined herein for the method according to the first aspect equally applies to the use according to the second aspect, and vice versa.

The bioconjugation reaction

The present invention revolves around a bioconjugation reaction. Bioconjugation reactions are well-known in the art and concern the covalent connection of one or more payloads to a biomolecule. In the context of the present invention, click probe Q forms a covalent attachment to click probe F on the biomolecule. Such conjugation reactions with alkyne and alkene moieties Q are well-known in the art as click reactions (see e.g. from WO 2014/065661 and Nguyen and Prescher, Nature rev. 2020, doi: 10.1038/s41570-020-0205-0, both incorporated by reference) and may typically take the form of a 1,3-dipolar cycloaddition or (4+2) cycloaddition. The alkyne-azide cycloaddition may be strain-promoted (e.g. a strain-promoted alkyne—azide cycloaddition, SPAAC), or may be catalysed (e.g. by copper), both of which are well-known. In a preferred embodiment, the bioconjugation reaction is a metal-free strain-promoted cycloaddition, most preferably metal-free strain-promoted alkyne-azide cycloaddition.

The bioconjugation reaction according to the present invention involves the reaction of (i) a cyclic alkyne or alkene compound of structure Q—L—D (2) , wherein Q comprises a cyclic alkyne or alkene moiety, L is a linker and D is a payload, with (ii) a molecule of structure B—(F)ₓ (3) , wherein

B is a biomolecule that is functionalized with x click probes F, and x is an integer in the range of 1 — 10, to form a bioconjugate of structure B—(Z—L—D)ₓ (1) , wherein the payload D is covalently attached to the biomolecule B via connecting group Z formed by the click reaction between Q and F. Herein, one molecule of structure B—(F)ₓ (3) reacts with x molecules of structure Q—L—D (2) . The bioconjugation reaction is performed in the presence of a surfactant.

The biomolecule is thus functionalized with x reactive groups F that are reactive towards an alkyne or an alkene in a cycloaddition, or in other words that is capable of forming a covalent attachment with an alkyne or alkene moiety. The skilled person is aware of such reactive groups, which may be selected from azide, tetrazine, triazine, nitrone, nitrile oxide, nitrile imine, diazo compound, ortho-quinone, dioxothiophene and sydnone. Preferred structures for the reactive group are structures (F1)-(F10) depicted here below.

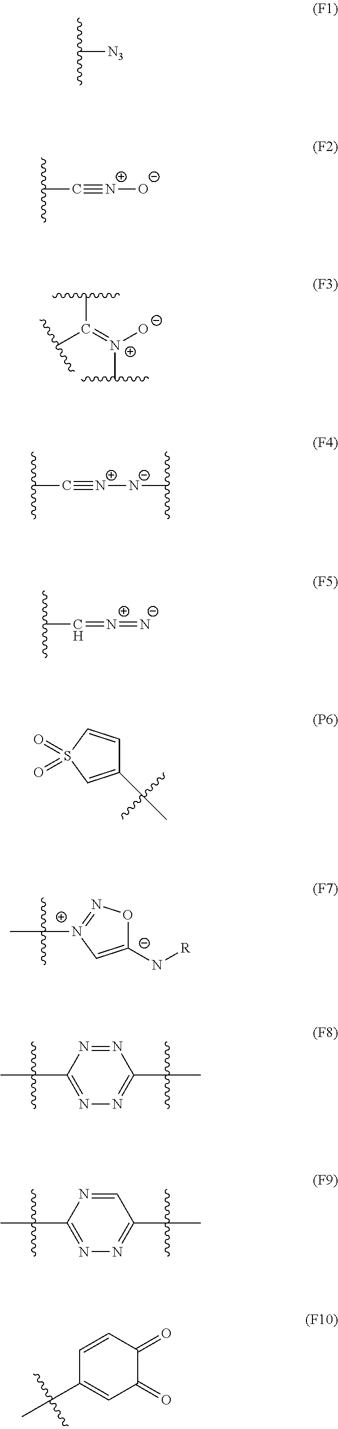

Herein, the wavy bond represents the connection to the biomolecule. For (F3), (F4), (F8) and (F9), the biomolecule can be connected to any one of the wavy bonds. The other wavy bond may then be connected to an R group selected from hydrogen, C₁-C₂₄ alkyl groups, C₂-C₂₄ acyl groups, C₃-C₂₄ cycloalkyl groups, C₂-C₂₄ (hetero)aryl groups, C₃-C₂₄ alkyl(hetero)aryl groups, C₃-C₂₄ (hetero)arylalkyl groups and C₁-C₂₄ sulfonyl groups, each of which (except hydrogen) may optionally be substituted and optionally interrupted by one or more heteroatoms selected from O, S and NR³² wherein R³² is independently selected from the group consisting of hydrogen and C₁-C₄ alkyl groups. The skilled person understands which R groups may be applied for each of the click probes F. For example, the R group connected to the nitrogen atom of (F3) may be selected from alkyl and aryl, and the R group connected to the carbon atom of (F3) may be selected from hydrogen, alkyl, aryl, acyl and sulfonyl. Preferably, click probe F is selected from azides or tetrazines. Most preferably, click probe F is an azide.

The nature of the biomolecule is not limited in the context of the present reaction. In a preferred embodiment, the biomolecule is selected from the group consisting of proteins (including glycoproteins such as antibodies), polypeptides, peptides, glycans, lipids, nucleic acids, oligonucleotides, polysaccharides, oligosaccharides, enzymes, hormones, amino acids and monosaccharides. More preferably from the group consisting of proteins, polypeptides, peptides and glycans. Preferably, the biomolecule contains a polypeptide part. An especially preferred class of biomolecules are glycoproteins, such as antibodies, which combine a hydrophilic peptide chain with a hydrophilic sugar chain (glycan). In a preferred embodiment, the biomolecule is selected from the group consisting of antibodies, Fab, VHH, scFv, diabody, minibody, affibody, affylin, affimers, atrimers, fynomer, Cys-knot, DARPin, adnectin/centryin, knottin, anticalin, FN3, Kunitz domain, OBody, bicyclic peptides and tricyclic peptides.

The biomolecule is preferably characterized as hydrophilic and/or water-soluble. The hydrophilic nature of the azide particularly surfaces when the azide moiety is connected to a monosaccharide moiety of a glycan of a glycoprotein. Most conveniently, the azide moiety is attached to a monosaccharide moiety of a glycan, preferably to the terminal monosaccharide moiety of a glycan of a glycoprotein, most preferably of an antibody.

x represents the amount of click probes F present on the biomolecule of structure (3), and is an integer in the range of 1-10. Preferably, x is an integer in the range of 1-8, more preferably x=1, 2, 3 or 4, even more preferably x=1 or 2, most preferably x=2. The bioconjugate of structure (1) normally has the same amount of moieties Z—L—D connected to the biomolecule, although the bioconjugation reaction may at times be slightly incomplete. Notably, each linker L may contain more than one payload D, such as 1 or 2 payload molecules per linker L.

Z is a connecting group. The term “connecting group” refers to a structural element connecting one part of the bioconjugate and another part of the same bioconjugate. In (1), Z connects biomolecule B with payload D, via linker L. As the skilled person understands, the exact nature of Z depends on the nature of F and Q. Preferred embodiments for Q are defined further below, but it contains at least a cyclic alkyne or alkene moiety. Preferably, Q contains a cyclic alkyne moiety. Connecting group Z may contain a triazole moiety, an isoxazole moiety, a dihydroisoxazole moiety, a bicyclo[2.2.2]octa-5,7-diene-2,3-dione moiety, a bicyclo[2.2.2]octa-5-ene-2,3-dione moiety, a 7-thiabicyclo[2.2.1]hepta-2,5-diene-7,7-dioxide moiety, a 7-thiabicyclo[2.2.1 ]hept-2-ene-7,7-dioxide moiety, a pyrazole moiety, a pyridine moiety, a dihydropyridine moiety, a pyridazine moiety ora dihydropyridazine moiety. Preferred structures for the connecting group Z are structures (Z1)-(Z8) depicted here below.

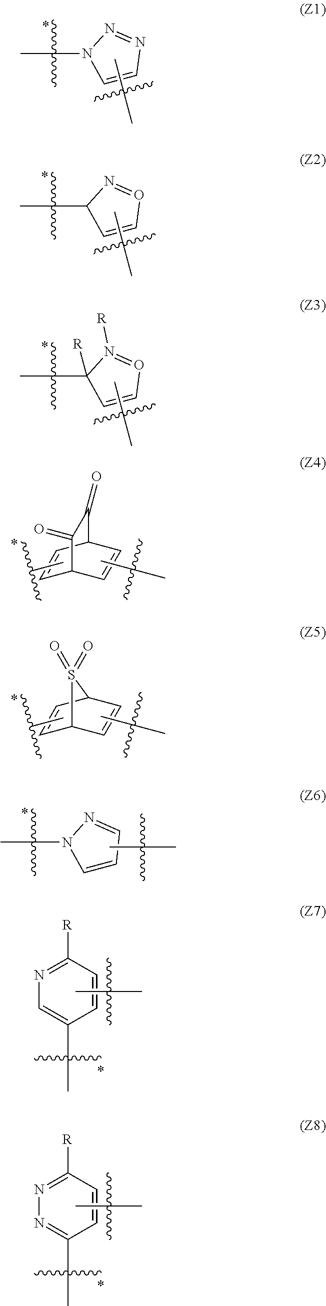

Herein, functional groups R in (Z3), (Z7) and (Z8) may be selected from hydrogen, C₁-C₂₄ alkyl groups, C₂-C₂₄ acyl groups, C₃-C₂₄ cycloalkyl groups, C₂-C₂₄ (hetero)aryl groups, C₃ -C₂₄ alkyl(hetero)aryl groups, C₃-C₂₄ (hetero)arylalkyl groups and C₁-C₂₄ sulfonyl groups, each of which (except hydrogen) may optionally be substituted and optionally be interrupted by one or more heteroatoms selected from O, S and NR³² wherein R³² is independently selected from the group consisting of hydrogen and C₁-Ca alkyl groups. The wavy bond labelled with an*is connected to the biomolecule and the other wavy bond to D. The skilled person understands which R groups may be applied for each of the connecting groups Z. For example, the R group connected to the nitrogen atom of (Z3) may be selected from alkyl or aryl as defined above, and the R group connected to the carbon atom of (Z3) may be selected from hydrogen, alkyl, aryl, acyl and sulfonyl as defined above.

In an especially preferred embodiment, Q contains a cyclic alkyne moiety and F is an azide, and Z contains a triazole moiety that is formed by 1,3-dipolar cycloaddition of the alkyne moiety with the azide moiety.

As explained above, the utilization of the surfactant according to the invention provides several unexpected advantages for the bioconjugation reaction. Hence, the use according to the second aspect may be for one or more of (i) increasing the conversion of the bioconjugation reaction; (ii) increasing the yield of the bioconjugation reaction; (iii) reducing the amount of organic co-solvent in the solvent system wherein the bioconjugation reaction is performed; (iv) providing flexibility in the concentration of biomolecule during the bioconjugation reaction; (v) reducing the excess of alkyne- or alkene-functionalized payload used during the bioconjugation reaction; (vi) reducing the extent of aggregate formation during the bioconjugation reaction and (vii) simplifying downstream processing of the bioconjugate. In one embodiment, the use according to the second aspect is at least for increasing the conversion of the bioconjugation reaction. In one embodiment, the use according to the second aspect is at least for increasing the yield of the bioconjugation reaction. In one embodiment, the use according to the second aspect is at least for reducing the amount of organic co-solvent in the solvent system wherein the bioconjugation reaction is performed. In one embodiment, the use according to the second aspect is at least for providing flexibility in the concentration of biomolecule during the bioconjugation reaction. In one embodiment, the use according to the second aspect is at least for reducing the excess of cyclic alkyne- or alkene-functionalized payload used during the bioconjugation reaction. In one embodiment, the use according to the second aspect is at least for reducing the extent of aggregate formation during the bioconjugation reaction. In one embodiment, the use according to the second aspect is at least for simplifying downstream processing of the bioconjugate.

As shown in the examples, the conversion of the bioconjugation reaction is improved when the surfactant is present in the reaction mixture. This in turn leads to a higher yield of the conjugate, but also to an improved drug-to-antibody ratio (DAR). The DAR of the obtained conjugates is closer to the theoretical DAR determined by the number of conjugation sites on the biomolecule and the number of payloads per conjugation site. Thus, the use according to the second aspect of the invention is in an especially preferred embodiment for improving the DAR, more in particular for obtaining a DAR that is close to the theoretical DAR. The DAR being closer to theoretical may refer to the average DAR of the obtained conjugate being closer to the absolute value of the theoretical DAR, but also the average DAR of the obtained conjugate having a lower standard deviation, even if the average DAR would be equally far from the theoretical DAR. The latter is especially relevant when conjugates with a theoretical DAR of 1 are prepared, when a mixture of DARO and DAR2 conjugates may give an average DAR close to theoretical, but with a large standard deviation. The use of the surfactant provides DAR1 conjugates with a low standard deviation, as shown in Examples 15 and 16.

Such improved DARs are obtained without changing the stoichiometry of the reaction partners (alkyne or alkene compound of structure (2) and molecule of structure (3)) in the reaction mixture. Conjugates having a closer to theoretical DAR are desirable, because they are more homogeneous, i.e. do not have a wide stochastic distribution of conjugates with different DARs deviating from the theoretical DAR, including lower-than-theoretical DAR and higher-than-theoretical DAR species. First of all, a conjugate with a low homogeneity will contain a large number of components, which does not only compromise analytics (disadvantageous from a regulatory standpoint) but will also contain components that will not or to a lesser extent contribute to the desired mode-of-action of the conjugate, or potentially even negatively impact the effectivity. For example, antibody-conjugates (i.e. having an antibody as biomolecule) with a low DAR will compete for the target receptor with the preferred conjugate with a higher DAR, but due to the lower-than-optimal number of drugs on the conjugate, a sufficient concentration of effective catabolite in the cell may potentially not be reached. Further, antibody-conjugates with a higher-than-optimal DAR and containing a hydrophobic payload (as most cytotoxic payloads) may rapidly be eliminated from circulation and possibly lead to enhance liver toxicity. Such disadvantages of antibody conjugates with cytotoxic drugs are well-known for many marketed ADCs, such as Adcetris®, Kadcyla® and others.

The utilization of the surfactant according to the invention enables the use of a lower amount of organic co-solvent in the solvent system wherein the bioconjugation reaction is performed. The amount of organic co-solvent could be reduced by 10 to 50 )/0, compared to the same reaction performed in the absence of surfactant. For increased stability of the antibody and decreased aggregation issues during the bioconjugation reaction, it is preferred that the bioconjugation reaction is performed in as little as possible organic solvent. However, for solubility reasons, the use of organic solvent can normally not be avoided completely. In the context of the present invention, the amount of organic solvent in the solvent system can be as low as 0-30 vol %. Thus, in a preferred embodiment, the reaction is performed in a solvent system containing water and organic solvent in a ratio in the range of 50/50-100/0 (v/v), preferably in the range of 75/25-95/5 (v/v). Although any organic solvent suitable for performing a bioconjugation reaction could be used, the organic solvent is preferably selected from dimethyl sulfoxide (DMSO), N,N-dimethylaniline (DMA), dimethylformamide (DMF), propylene glycol (PG), pyridine and N-methyl-2-pyrrolidone

(NMP). Most preferably, the organic solvent is DMF or PG.

The surfactant usage according to the invention thus leads to reduced aggregate formation during the bioconjugation process, such as aggregation below 10%, preferably below 5%, more preferably below 3%, most preferably below 1%. Typically, these values show a decrease in aggregation of at least 20 )/0, more preferably at least 30%, most preferably at least 50%, compared to the same reaction in the absence of surfactant. Extent of aggregation can readily be determined by size exclusion chromatography on a sample of the reaction mixture.

The utilization of the surfactant according to the invention furthermore provides flexibility in the concentration of the functionalized biomolecule of structure (3) that can be used during the bioconjugation reaction. Such flexibility may take the form of an increase or a decrease, both of which can be advantageous depending on the exact nature of the biomolecule. For example, the surfactant enables the use of a higher concentration of the functionalized biomolecule of structure (3). In terms of reaction kinetics, it is preferred that the concentration of the biomolecule is as high as possible. Thus, in a preferred embodiment, the concentration of the azide-functionalized biomolecule is in the range of 1-100 mg/mL, preferably in the range of 5-50 mg/mL, more preferably in the range of 8-25 mg/mL, most preferably in the range of 10-20 mg/mL.

The utilization of the surfactant according to the invention furthermore enables the use of less excess of functionalized payload during the bioconjugation reaction. A reduction of at least 20% , even up to a reduction of 50% or more, of functionalized payload of structure (2) could be accomplished, when compared to the same bioconjugation reaction in absence of the surfactant.

As cyclic alkyne- and alkene-functionalized payloads are typically expensive and laborious to make, the invention improves the overall (cost) efficacy of the bioconjugation process. Thus, in a preferred embodiment, the functionalized payload of structure (2) is present in at most 5-fold excess, preferably in at most 3-fold excess, more preferably in at most 2-fold excess, most preferably in at most 1.5-fold excess with respect to the functionalized biomolecule. The stoichiometry of the functionalized payload of structure (2) should typically be at least 1, such that one molecule of functionalized payload is available per click probe F. The present invention provides optimal conjugation reaction with stoichiometry of the functionalized payload close to 1. From a practical point of view, the stoichiometry may be slightly above 1, such as at least 1.1 or at least 1.2. As common in the art, the excess is determined stoichiometrically. Thus, in case x=2 and the excess=2-fold, then the bioconjugation reaction is performed with 4 moles of functionalized payload of structure (2) per mole of biomolecule of structure (3).

The use of less co-solvent and a smaller excess functionalized payload during the bioconjugation reaction simplifies the downstream processing of the bioconjugate. Herein, downstream processing typically refers to isolation and/or purification of the bioconjugate, such that it can be used in a clinical setting. For example, a filtration step to remove smaller molecules from the bioconjugate can be reduced or completely obviated. In other words, the manufacturing of a suitable medicament from the thus formed bioconjugate is simplified.

A further advantage of the use of surfactants in bioconjugation reactions using click probes is that the number of conjugation sites on the biomolecule is not reduced, and the relative distribution is unchanged, contrary to what was observed for bioconjugation via acylated lysine technology in the presence of surfactants. In the present invention, the number of conjugation sites, and thus the theoretical drug-to-antibody ratio (DAR), is governed by the amount of click probes F (i.e. the value x) present on the biomolecule. The presence of surfactant during the bioconjugation reaction provides for an improved reaction efficacy, as explained above, but does not provide a different product. Thus, in the context of the present invention, there will be no difference in production of the biomolecule conjugate during early development and production of the same biomolecule conjugate after process optimization. In addition, the surfactant can be omitted, if needed for some reason (for example lack of availability), without changing the structure of the final bioconjugate.

### The Surfactant

The present invention utilizes a surfactant. Surfactants are a well-known in the art. In a preferred embodiment, the surfactant contains at least a negatively charged group, i.e. is anionic or zwitterionic. Most preferably, the surfactant is anionic. Surprisingly superior results have been obtained with anionic surfactants. For anionic surfactant, the counter-ion is preferably an alkali metal cation, preferably Na. Zwitterionic surfactants may also have counter-ions (positively and negatively charged), but usually they balance their own charge without the need for counter-ions. The negatively charged group is preferably selected from sulfate, carboxylate and phosphate. The positively charged group, if present, is preferably ammonium.

In a preferred embodiment, the surfactant has structure R⁴-X, wherein R⁴ is selected from long chain alkyl, alkylaryl and cholane derivatives, wherein the alkyl and alkylaryl moieties may optionally be fluorinated, and X is COO⁽⁻⁾, 50₃⁽⁻⁾ or PO₃⁽²⁻⁾. Preferably, X is COOH. In the context of R⁴, the alkyl is preferably C₈-C₁₀₀ alkyl moiety, preferably a C₉-C₅₀ alkyl moiety, more preferably a C₁₀-C₂₄ alkyl moiety. In an especially preferred embodiment, R⁴ is C₁₀-C₁₂ alkyl or cholane and X is COO^{(-).}

Alternatively, the surfactant may be selected from the group consisting of decanoate, dodecanoate, dodecyl sulfate (e.g. SDS), deoxycholate, 3-[(3-cholamidopropyl)dimethylammonio]-1-propanesulfonate (CHAPS) and 3-[(3-cholamidopropyl)dimethylammonio]-2-hydroxy propanesulfonate (CHAPSO), preferably wherein the surfactant is decanoate, dodecanoate or deoxycholate, more preferably the surfactant is decanoate or deoxycholate, most preferably the surfactant is deoxycholate. In one embodiment, the surfactant is sodium decanoate. In another embodiment, the surfactant is sodium deoxycholate.

The alkyne- or alkene-functionalized payload of structure Q—L—D (2)

The alkyne or alkene compound according to the present invention has the structure Q—L—D (2), wherein:

Q comprises a cyclic alkyne or a cyclic alkene moiety,

L is a linker, and

D is a payload.

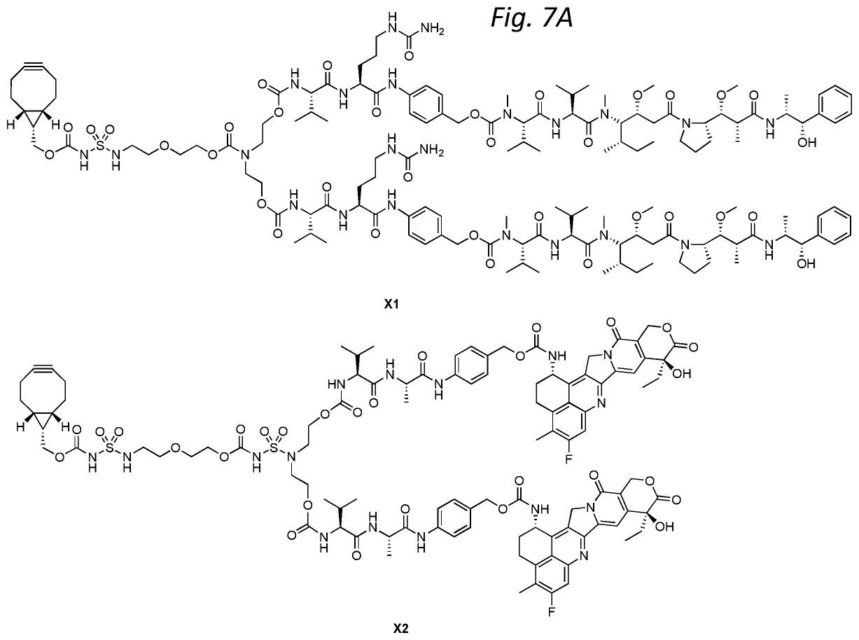

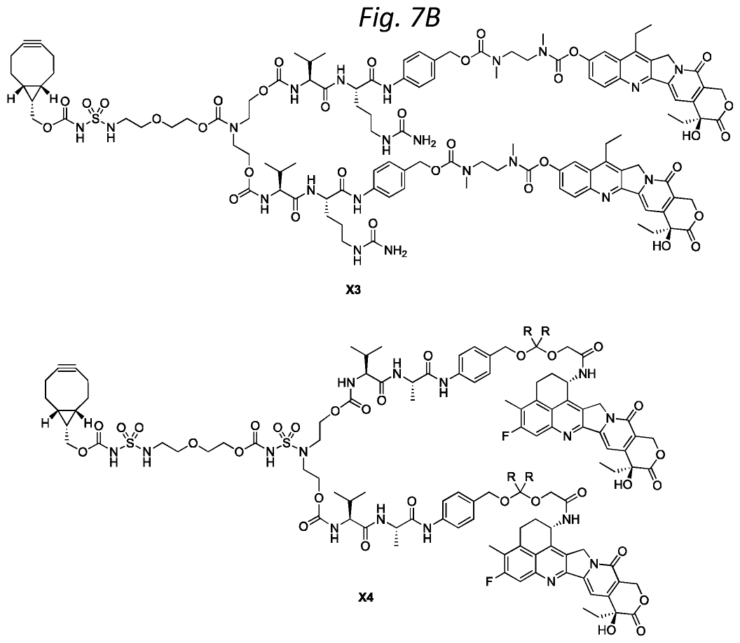

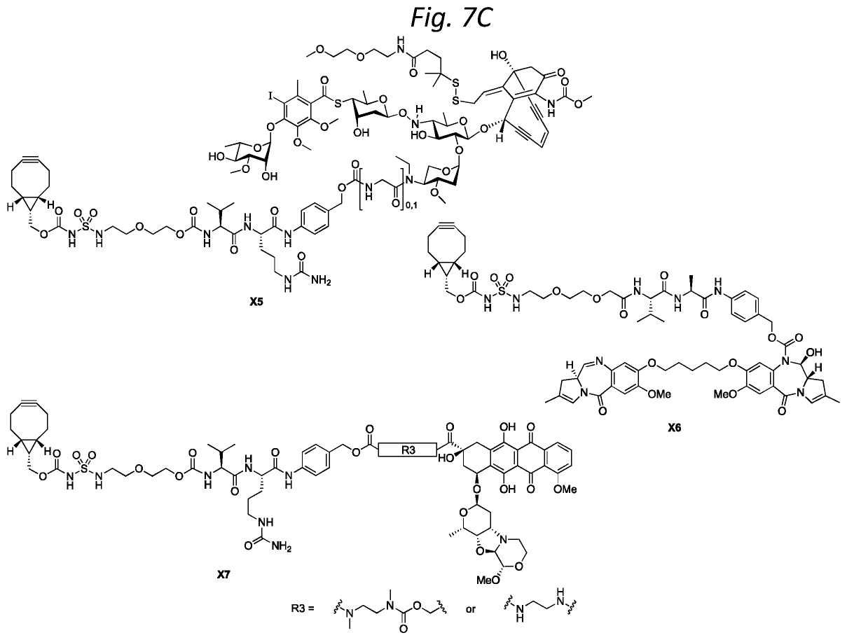

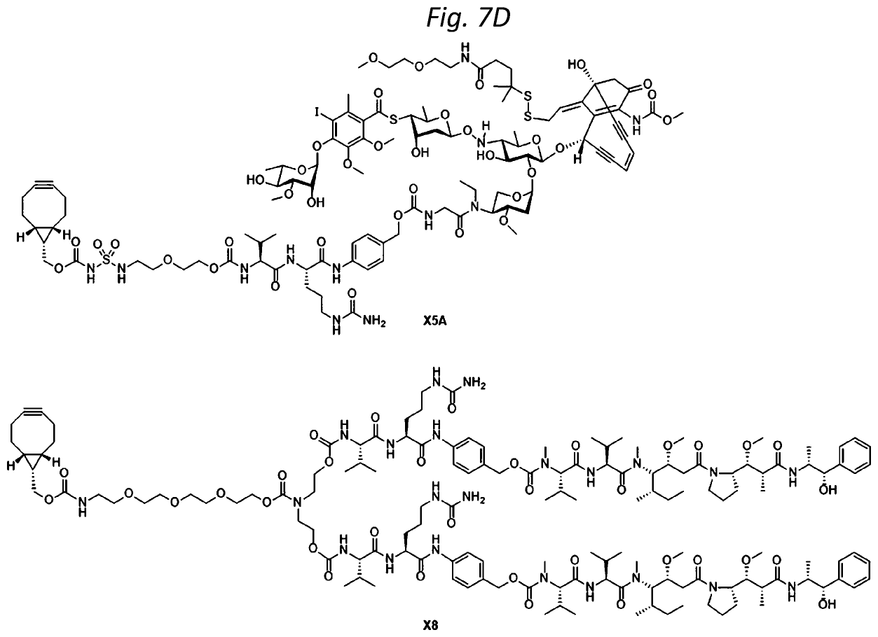

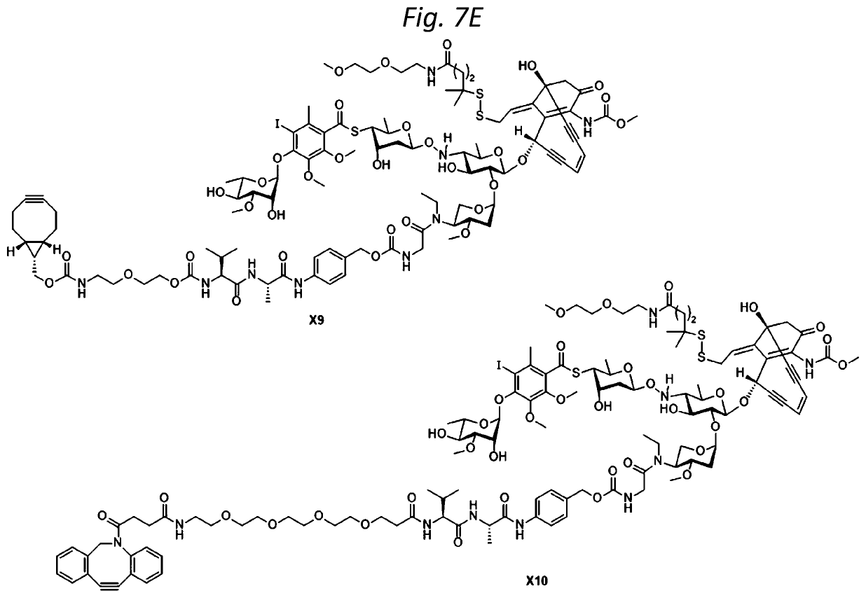

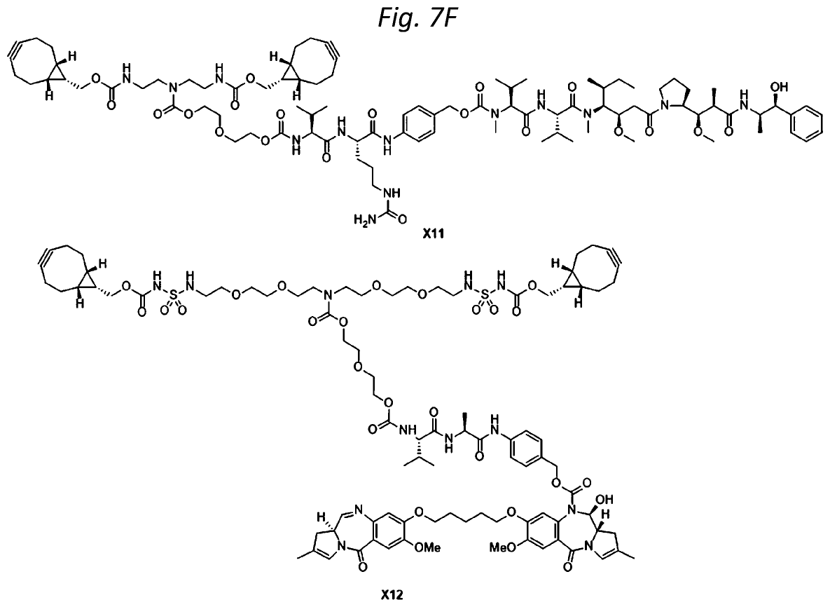

Here below, each of Q, L and D is further described. Preferred embodiments of the of the alkyne or alkene compound having structure (2) are described further below. Especially preferred embodiments, wherein Q comprises a cyclic alkyne, are depicted in FIGS. 7A-7F, even more preferably in FIGS. 7A-7C.

Click Probe Q: Cyclic Alkyne or Alkene

Click probe Q is used in the bioconjugation process to connect the alkyne- or alkene-payload construct to the biomolecule of structure B—(F)ₓ (3). Q may be a cyclic alkene or a cyclic alkyne moiety, which are both reactive towards click probe F in a click reaction. Preferably, Q is an cyclic alkyne moiety.

In an especially preferred embodiment, the click probe Q comprises a cyclic alkyne moiety. The alkynyl group may also be referred to as a (hetero)cycloalkynyl group, i.e. a heterocycloalkynyl group or a cycloalkynyl group, wherein the (hetero)cycloalkynyl group is optionally substituted. Preferably, the (hetero)cycloalkynyl group is a (hetero)cycloheptynyl group, a (hetero)cyclooctynyl group, a (hetero)cyclononynyl group or a (hetero)cyclodecynyl group. Most preferably, the (hetero)cycloalkynyl group is a (hetero)cyclooctynyl group, wherein the (hetero)cyclooctynyl group is optionally substituted. Herein, the alkynes and (hetero)cycloalkynes may optionally be substituted. Preferably, Q comprises a (hetero)cyclooctyne moiety according to structure (Q1) below. In a further preferred embodiment, the (hetero)cyclooctynyl group is according to structure (Q37), (Q38) or (Q39) as defined further below. Preferred examples of the (hetero)cyclooctynyl group include structure (Q2), also referred to as a DIBO group, (Q3), also referred to as a DIBAC group, or (Q4), also referred to as a BARAC group, (Q5), also referred to as a COMBO group, and (Q6), also referred to as a BCN group, all as shown below, wherein Y¹ is O or NR¹¹, wherein R¹¹ is independently selected from the group consisting of hydrogen, a linear or branched C₁-C₁₂ alkyl group or a C₄-C₁₂ (hetero)aryl group. The aromatic rings in (Q2) are optionally O-sulfonylated at one or more positions, whereas the rings of (Q3) and (Q4) may be halogenated at one or more positions. A particularly preferred cycloalkynyl group is a bicyclo[6.1.0]non[-4-yn-9-yl] group (BCN group), which is optionally substituted. Preferably, the bicyclo[6.1.0]non-4-yn[-9-yl] group is according to formula (Q6) as shown below, wherein V is (CH₂)₁ and I is an integer in the range of 0 to 10, preferably in the range of 0 to 6. More preferably, I is 0, 1, 2, 3 or 4, more preferably I is 0, 1 or 2 and most preferably I is 0 or 1. In the context of group (Q6), I is most preferably 1.

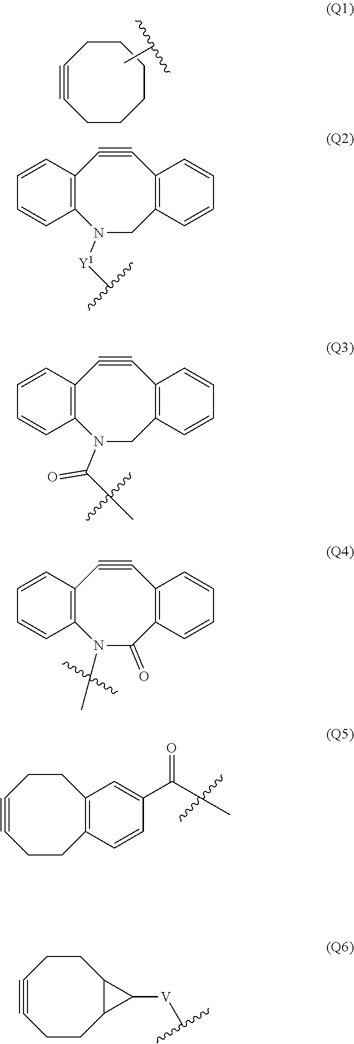

In a further preferred embodiment, the click probe Q is selected from the group consisting of (Q7)-(Q21) depicted here below.

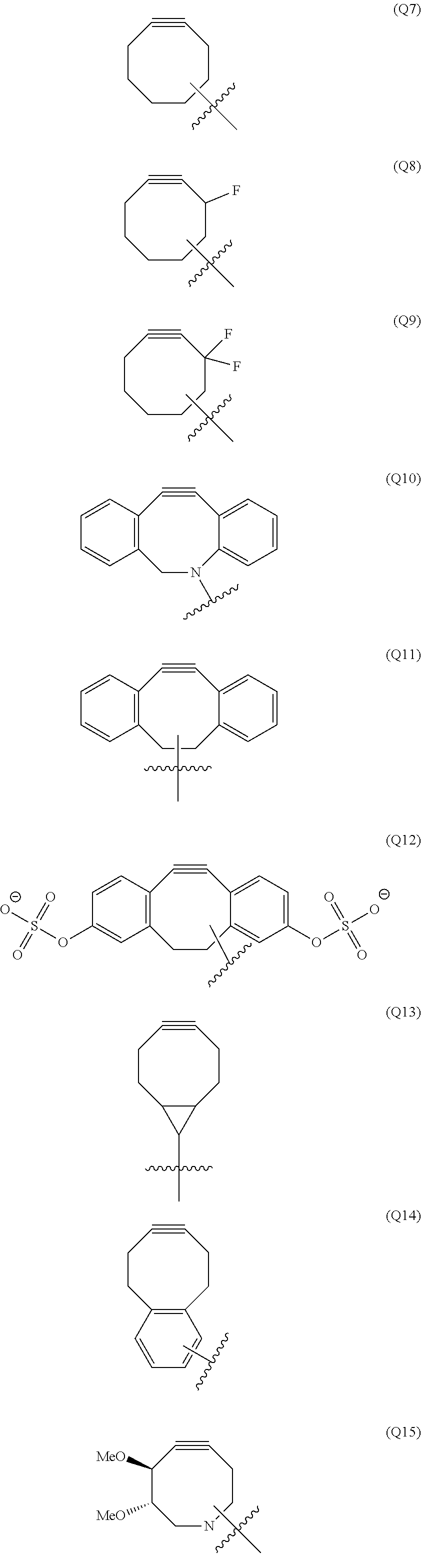

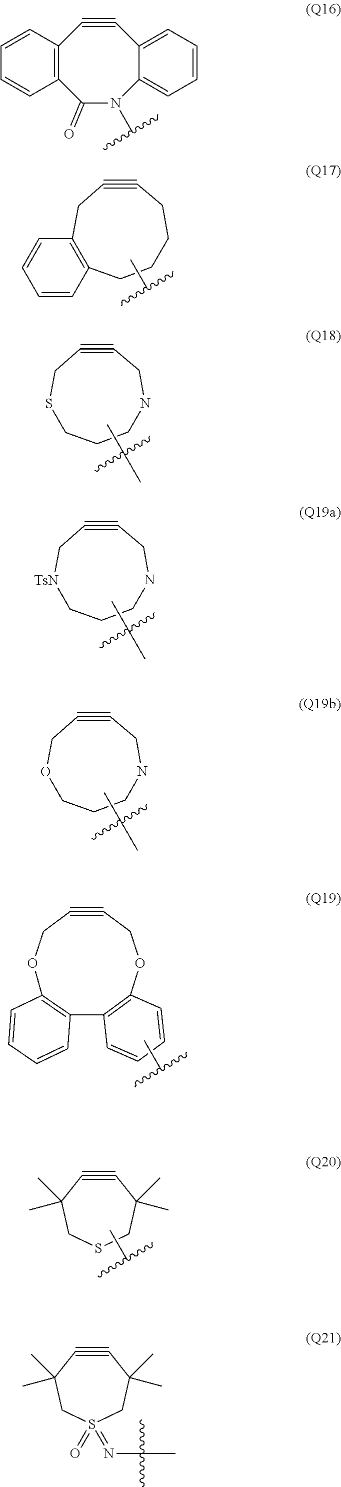

Herein, the connection to L, depicted with the wavy bond, may be to any available carbon or nitrogen atom of Q.

In a further preferred embodiment, the click probe Q is selected from the group consisting of (Q22)-(Q36) depicted here below.

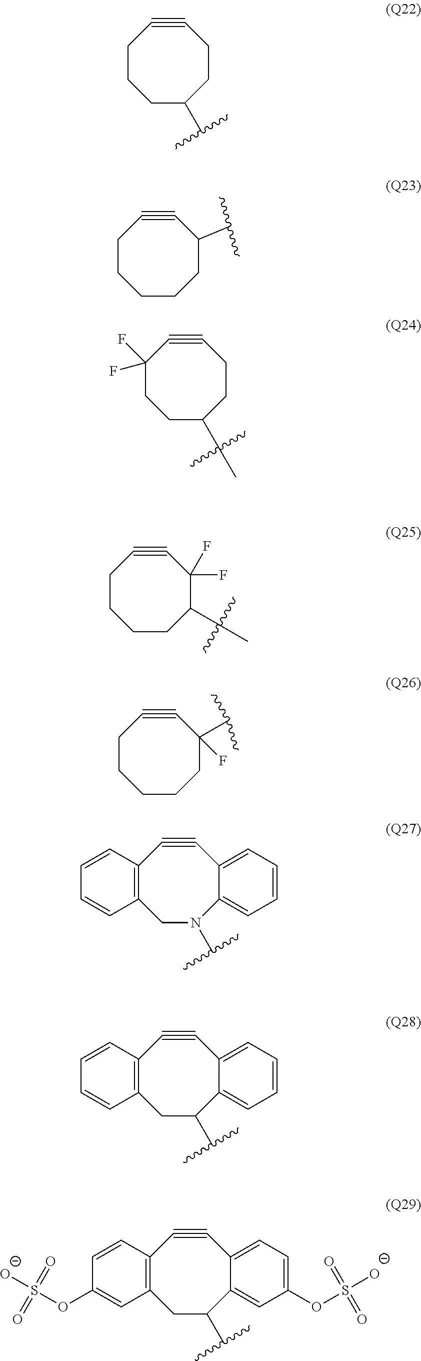

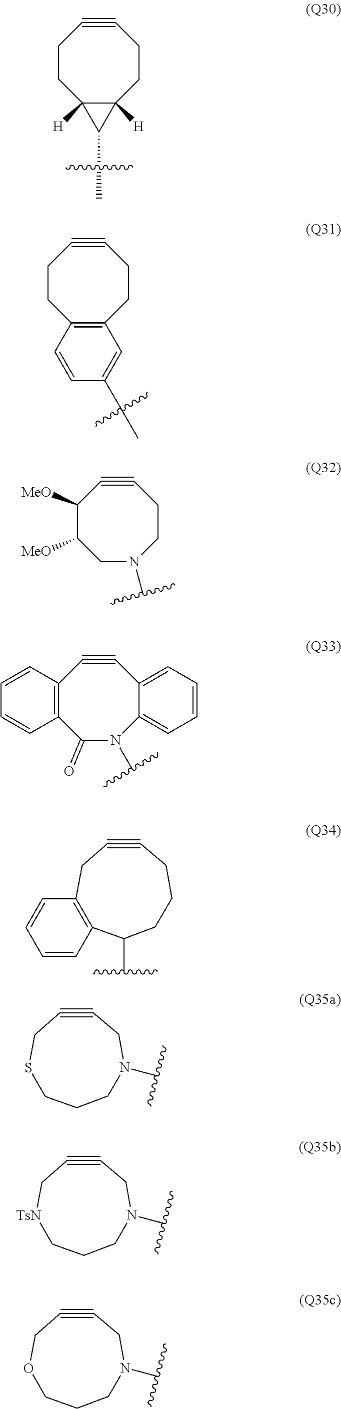

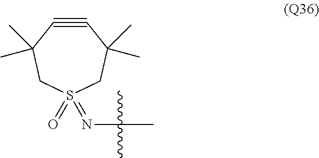

In an especially preferred embodiment, click probe Q comprises an (hetero)cycloalkynyl group and is according to structure (Q37):

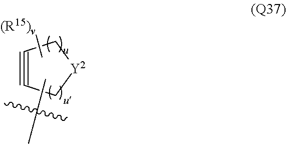

Herein:
R¹⁵ is independently selected from the group consisting of hydrogen, halogen, —OR¹⁶, —NO₂, —CN, —S(O)₂R¹⁶, —S(O)₃⁽⁻⁾, C₁-C₂₄ alkyl groups, C₆-C₂₄ (hetero)aryl groups, C₇-C₂₄ alkyl(hetero)aryl groups and C₇-C₂₄ (hetero)arylalkyl groups and wherein the alkyl groups, (hetero)aryl groups, alkyl(hetero)aryl groups and (hetero)arylalkyl groups are optionally substituted, wherein two substituents R¹⁵ may be linked together to form an optionally substituted annulated cycloalkyl or an optionally substituted annulated (hetero)arene substituent, and wherein R¹⁶ is independently selected from the group consisting of hydrogen, halogen, C₁-C₂₄ alkyl groups, C₆-C₂₄ (hetero)aryl groups, C₇-C₂₄ alkyl(hetero)aryl groups and C₇-C₂₄ (hetero)arylalkyl groups;

Y² is C(R³¹)₂, O, S or NR³¹, wherein each R³¹ individually is R¹⁵ or —LD;

u is 0, 1, 2, 3, 4 or 5;

u′ is 0, 1, 2, 3, 4 or 5, wherein u+u′=4, 5, 6, 7 or 8;

v=an integer in the range 8-16.

In a preferred embodiment, u +u′=4, 5 or 6, more preferably u+u′=5. Typically, v=(u+u′)×2 or [(u+u′)×2]−1. In a preferred embodiment, v=8, 9 or 10, more preferably v=9 or 10, most preferably v=10.

In an especially preferred embodiment, click probe Q comprises an alkynyl group and is according to structure (Q38):

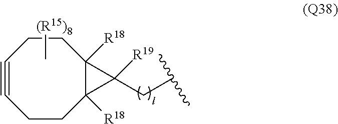

Herein:
R¹⁵ is independently selected from the group consisting of hydrogen, halogen, —OR¹⁶, —NO₂, —CN, —S(O)₂R¹⁶, —S(O)₃⁽⁻⁾,C₁-C₂₄ alkyl groups, Cs-C₂₄ (hetero)aryl groups, C₇-C₂₄ alkyl(hetero)aryl groups and C₇-C₂₄ (hetero)arylalkyl groups and wherein the alkyl groups, (hetero)aryl groups, alkyl(hetero)aryl groups and (hetero)arylalkyl groups are optionally substituted, wherein two substituents R¹⁵ may be linked together to form an optionally substituted annulated cycloalkyl or an optionally substituted annulated (hetero)arene substituent, and wherein R¹⁶ is independently selected from the group consisting of hydrogen, halogen, C₁-C₂₄ alkyl groups, C₆-C₂₄ (hetero)aryl groups, C₇-C₂₄ alkyl(hetero)aryl groups and C₇-C₂₄ (hetero)arylalkyl groups;
R¹⁸ is independently selected from the group consisting of hydrogen, halogen, C₁-C₂₄ alkyl groups, C₆-C₂₄ (hetero)aryl groups, C₇-C₂₄ alkyl(hetero)aryl groups and C₇-C₂₄ (hetero)arylalkyl groups;
R¹⁹ is selected from the group consisting of hydrogen, —LD; halogen, C₁-C₂₄ alkyl groups, C₆-C₂₄ (hetero)aryl groups, C₇-C₂₄ alkyl(hetero)aryl groups and C₇-C₂₄ (hetero)arylalkyl groups, the alkyl groups optionally being interrupted by one of more hetero-atoms selected from the group consisting of O, N and S, wherein the alkyl groups, (hetero)aryl groups, alkyl(hetero)aryl groups and (hetero)arylalkyl groups are independently optionally substituted; and
I is an integer in the range 0 to 10.

In a preferred embodiment of the reactive group according to structure (Q38), R¹⁵ is independently selected from the group consisting of hydrogen, halogen, —OR¹⁶,-C₆ alkyl groups, C₅-C₆ (hetero)aryl groups, wherein R¹⁶ is hydrogen or C₁-C₆ alkyl, more preferably R¹⁵ is independently selected from the group consisting of hydrogen and C₁-C₆ alkyl, most preferably all R¹⁵ are H. In a preferred embodiment of the reactive group according to structure (Q38), R¹⁸ is independently selected from the group consisting of hydrogen, C₁-C₆ alkyl groups, most preferably both R¹⁸ are H. In a preferred embodiment of the reactive group according to structure (Q38), R¹⁹ is H. In a preferred embodiment of the reactive group according to structure (Q38), I is 0 or 1, more preferably I is 1. An especially preferred embodiment of the reactive group according to structure (Q38) is the reactive group according to structure (Q30).

In an especially preferred embodiment, click probe Q comprises an alkynyl group and is according to structure (Q39):

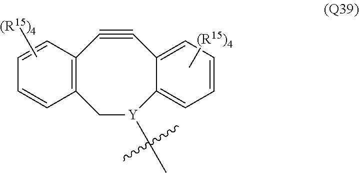

Herein:
R¹⁵ is independently selected from the group consisting of hydrogen, halogen, —OR¹⁶, —NO₂, —CN, —S(O)₂R¹⁶, —S(O)₃⁽⁻⁾, C₁-C₂₄ alkyl groups, C₅-C₂₄ (hetero)aryl groups, C₇-C₂₄ alkyl(hetero)aryl groups and C₇-C₂₄ (hetero)arylalkyl groups and wherein the alkyl groups, (hetero)aryl groups, alkyl(hetero)aryl groups and (hetero)arylalkyl groups are optionally substituted, wherein two substituents R¹⁵ may be linked together to form an optionally substituted annulated cycloalkyl or an optionally substituted annulated (hetero)arene substituent, and wherein R¹⁶ is independently selected from the group consisting of hydrogen, halogen, C₁-C₂₄ alkyl groups, C₆-C₂₄ (hetero)aryl groups, C₇-C₂₄ alkyl(hetero)aryl groups and C₇-C₂₄ (hetero)arylalkyl groups;
Y is N or CR¹⁵.

In a preferred embodiment of the reactive group according to structure (Q39), R¹⁵ is independently selected from the group consisting of hydrogen, halogen, —OR¹⁶, —S(O)₃⁽⁻⁾, C₁-C₆ alkyl groups, C₅-C₆ (hetero)aryl groups, wherein R¹⁶ is hydrogen or C₁-C₆ alkyl, more preferably R¹⁵ is independently selected from the group consisting of hydrogen and —S(O)₃⁰. In a preferred embodiment of the reactive group according to structure (Q39), Y is N or CH, more preferably Y =N.

In an alternative preferred embodiment, click probe Q comprises a cyclic alkene moiety. The alkenyl group Q may also be referred to as a (hetero)cycloalkenyl group, i.e. a heterocycloalkenyl group or a cycloalkenyl group, preferably a cycloalkenyl group, wherein the (hetero)cycloalkenyl group is optionally substituted. Preferably, the (hetero)cycloalkenyl group is a (hetero)cyclopropenyl group, a (hetero)cyclobutenyl group, a trans-(hetero)cycloheptenyl group, a trans-(hetero)cyclooctenyl group, a trans-(hetero)cyclononenyl group or a trans-(hetero)cyclodecynyl group, which may all optionally be substituted. Especially preferred are (hetero)cyclopropenyl groups, trans-(hetero)cycloheptenyl group or trans-(hetero)cyclooctenyl groups, wherein the (hetero)cyclopropenyl group, the trans-(hetero)cycloheptenyl group or the trans-(hetero)cyclooctynyl group is optionally substituted. Preferably, Q comprises a cyclopropenyl moiety according to structure (Q40), a trans-(hetero)cycloheptenyl moiety according to structure (Q41) or a trans-(hetero)cyclooctenyl moiety according to structure (Q42). In a further preferred embodiment, the cyclopropenyl group is according to structure (Q43). In another preferred embodiment, the trans-(hetero)cycloheptene group is according to structure (Q44) or (Q45). In another preferred embodiment, the trans-(hetero)cyclooctene group is according to structure (Q46), (Q47), (Q48), (Q49) or (Q50).

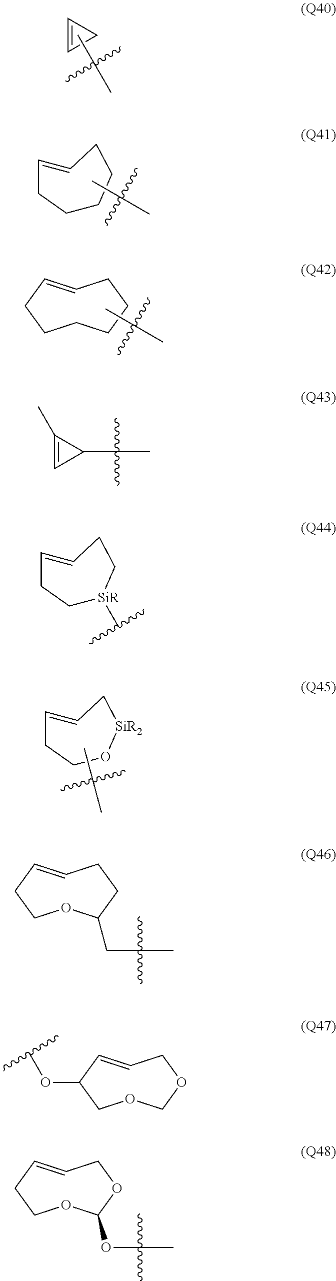

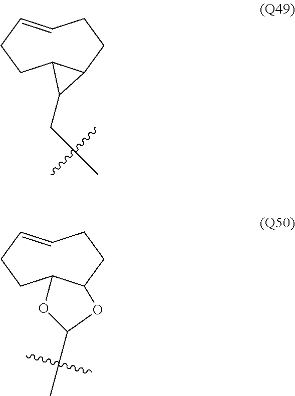 [SMILES: *CC1C2CC/C=C/CCC12 ; *C1OC2CC/C=C/CCC2O1]

Herein, the R group(s) on Si in (Q44) and (Q45) are typically alkyl or aryl, preferably C₁-Cs alkyl.

### Linker L

Linkers, also referred to as linking units, are well known in the art and any suitable linker may be used. In the final cyclic alkyne- or alkene-linker-payload construct, the payload is chemically connected to a cyclic alkene or alkyne via a cleavable or non-cleavable linker. The linker may contain one or more branch-points for attachment of multiple payloads to a single cyclic alkene or cyclic alkyne. Preparation of the cyclic alkyne- or alkene-linker-drug can be achieved by chemical methods described herein.

The linker may for example be selected from the group consisting of linear or branched C₁-C₂₀₀ alkylene groups, C₂-C₂₀₀ alkenylene groups, C₂-C₂₀₀ alkynylene groups, C₃-C₂₀₀ cycloalkylene groups, C₅-C₂₀₀ cycloalkenylene groups, C₈-C₂₀₀ cycloalkynylene groups, C₇-C₂₀₀ alkylarylene groups, C₇-C₂₀₀ arylalkylene groups, C₈-C₂₀₀ arylalkenylene groups, C₉-C₂₀₀ arylalkynylene groups. Optionally the alkylene groups, alkenylene groups, alkynylene groups, cycloalkylene groups, cycloalkenylene groups, cycloalkynylene groups, alkylarylene groups, arylalkylene groups, arylalkenylene groups and arylalkynylene groups may be substituted, and optionally said groups may be interrupted by one or more heteroatoms, preferably 1 to 100 heteroatoms, said heteroatoms preferably being selected from the group consisting of O, S(O)_{Y} and NR¹², wherein y is 0, 1 or 2, preferably y=2, and R¹² is independently selected from the group consisting of hydrogen, halogen, C₁-C₂₄ alkyl groups, C₆-C₂₄ (hetero)aryl groups, C₇-C₂₄ alkyl(hetero)aryl groups and C₇-C₂₄ (hetero)arylalkyl groups. The linker may contain (poly)ethylene glycoldiamines (e.g. 1,8-diamino-3,6-dioxaoctane or equivalents comprising longer ethylene glycol chains), (poly)ethylene glycol or (poly)ethylene oxide chains, (poly)propylene glycol or (poly)propylene oxide chains and 1,z-diaminoalkanes wherein z is the number of carbon atoms in the alkane, and may for example range from 2-25.

In a preferred embodiment, linker L comprises a sulfamide group, preferably a sulfamide group according to structure (L1):

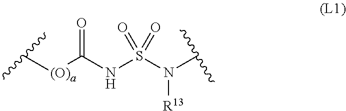

The wavy lines represent the connection to the remainder of the compound, typically to Q and to D, optionally via a spacer. Preferably, the (O)ₐC(O) moiety is connected to Q and the NR¹³ moiety to D.

In structure (L1), a=0 or 1, preferably a=1, and R¹³ is selected from the group consisting of hydrogen, C₁-C₂₄ alkyl groups, C₃-C₂₄ cycloalkyl groups, C₂-C₂₄ (hetero)aryl groups, C₃ -C₂₄ alkyl(hetero)aryl groups and C₃-C₂₄ (hetero)arylalkyl groups, the C₁-C₂₄ alkyl groups, C₃-C₂₄ cycloalkyl groups, C₂-C₂₄ (hetero)aryl groups, C₃-C₂₄ alkyl(hetero)aryl groups and C₃-C₂₄ (hetero)arylalkyl groups optionally substituted and optionally interrupted by one or more heteroatoms selected from O, S and NR¹⁴ wherein R¹⁴ is independently selected from the group consisting of hydrogen and C₁-C₄ alkyl groups, or R¹³ is a second occurrence of D connected to N via a spacer moiety, preferably Sp² as defined here below.

In a preferred embodiment, R¹³ is hydrogen or a C₁-C₂₀ alkyl group, more preferably R¹³ is hydrogen or a C₁- C₁₆ alkyl group, even more preferably R¹³ is hydrogen or a C₁-C₁₀ alkyl group, wherein the alkyl group is optionally substituted and optionally interrupted by one or more heteroatoms selected from O, S and NR¹⁴, preferably O, wherein R¹⁴ is independently selected from the group consisting of hydrogen and C₁-C₄ alkyl groups. In a preferred embodiment, R¹³ is hydrogen. In another preferred embodiment, R¹³ is a C₁-C₂₀ alkyl group, more preferably a C₁-C₁₆ alkyl group, even more preferably a C₁-C₁₀ alkyl group, wherein the alkyl group is optionally interrupted by one or more O-atoms, and wherein the alkyl group is optionally substituted with an —OH group, preferably a terminal —OH group. In this embodiment it is further preferred that R¹³ is a (poly)ethylene glycol chain comprising a terminal —OH group. In another preferred embodiment, R¹³ is selected from the group consisting of hydrogen, methyl, ethyl, n-propyl, i-propyl, n-butyl, s-butyl and t-butyl, more preferably from the group consisting of hydrogen, methyl, ethyl, n-propyl and i-propyl, and even more preferably from the group consisting of hydrogen, methyl and ethyl. Yet even more preferably, R¹³ is hydrogen or methyl, and most preferably R¹³ is hydrogen.

In a preferred embodiment, the linker is according to structure (L2):

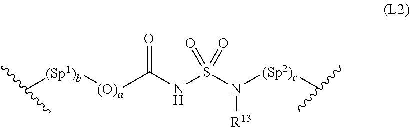

Herein, a, R¹³ and the wavy lines are as defined and Sp² are independently spacer moieties and b and c are independently 0 or 1. Preferably, b=0 or 1 and c=1. more preferably b=0 and c=1. In one embodiment, spacers Sp¹ and Sp² are independently selected from the group consisting of linear or branched C₁-C₂₀₀ alkylene groups, C₂-C₂₀₀ alkenylene groups. C₂-C₂₀₀ alkynylene groups. C₃-C₂₀₀ cycloalkylene groups. C₅-C₂₀₀ cycloalkenylene groups. C₈-C₂₀₀ cycloalkynylene groups, C₇-C₂₀₀ alkylarylene groups. C₇-C₂₀₀ arylalkylene groups, C₈-C₂₀₀ arylalkenylene groups and C₇-C₂₀₀ arylalkynylene groups, the alkylene groups, alkenylene groups, alkynylene groups, cycloalkylene groups, cycloalkenylene groups, cycloalkynylene groups, alkylarylene groups, arylalkylene groups, arylalkenylene groups and arylalkynylene groups being optionally substituted and optionally interrupted by one or more heteroatoms selected from the group of O, S and NR²⁰, wherein R²⁰ is independently selected from the group consisting of hydrogen, C₁-C₂₄ alkyl groups. C₂-C₂₄ alkenyl groups, C₂- C₂₄ alkynyl groups and C₃-C₂₄ cycloalkyl groups, the alkyl groups, alkenyl groups, alkynyl groups and cycloalkyl groups being optionally substituted. When the alkylene groups, alkenylene groups, alkynylene groups, cycloalkylene groups, cycloalkenylene groups, cycloalkynylene groups, alkylarylene groups, arylalkylene groups, arylalkenylene groups and arylalkynylene groups are interrupted by one or more heteroatoms as defined above, it is preferred that said groups are interrupted by one or more O-atoms, and/or by one or more S—S groups.

More preferably, spacer moieties Sp¹ and Sp², if present, are independently selected from the group consisting of linear or branched C₁-C₁₀₀ alkylene groups. C₂-C₁₀₀ alkenylene groups, C₂-C₁₀₀ alkynylene groups, C₃-C₁₀₀ cycloalkylene groups, C₃-C₁₀₀ cycloalkenylene groups, C₈-C₁₀₀ cycloalkynylene groups, C₇-C₁₀₀ alkylarylene groups, C₇-C₁₀₀ arylalkylene groups, C₈-C₁₀₀ arylalkenylene groups and C₉-C₁₀₀ arylalkynylene groups, the alkylene groups, alkenylene groups, alkynylene groups, cycloalkylene groups, cycloalkenylene groups, cycloalkynylene groups, alkylarylene groups, arylalkylene groups, arylalkenylene groups and arylalkynylene groups being optionally substituted and optionally interrupted by one or more heteroatoms selected from the group of O, S and NR²⁰, wherein R²⁰ is independently selected from the group consisting of hydrogen, C₁-C₂₄ alkyl groups, C₂-C₂₄ alkenyl groups, C₂-C₂₄ alkynyl groups and C₃-C₂₄ cycloalkyl groups, the alkyl groups, alkenyl groups, alkynyl groups and cycloalkyl groups being optionally substituted.

Even more preferably, spacer moieties Sp¹ and Sp². if present, are independently selected from the group consisting of linear or branched C₁-C₅₀ alkylene groups. C₂-C₅₀ alkenylene groups, C₂-C₅₀ alkynylene groups, C₃-C₅₀ cycloalkylene groups, C₇-C₅₀ cycloalkenylene groups, C₈-C₅₀ cycloalkynylene groups, C₇-C₅₀ alkylarylene groups, C₇-C₅₀ arylalkylene groups, C₈-C₅₀ arylalkenylene groups and C₉-C₅₀ arylalkynylene groups, the alkylene groups, alkenylene groups, alkynylene groups, cycloalkylene groups, cycloalkenylene groups, cycloalkynylene groups, alkylarylene groups, arylalkylene groups, arylalkenylene groups and arylalkynylene groups being optionally substituted and optionally interrupted by one or more heteroatoms selected from the group of O, S and NR²⁰, wherein R₂₀ is independently selected from the group consisting of hydrogen, C₁-C₂₄ alkyl groups, C₂-C₂₄ alkenyl groups, C₂-C₂₄ alkynyl groups and C₃-C₂₄ cycloalkyl groups, the alkyl groups, alkenyl groups, alkynyl groups and cycloalkyl groups being optionally substituted.

Yet even more preferably, spacer moieties Sp¹ and Sp², if present, are independently selected from the group consisting of linear or branched C₁-C₂₀ alkylene groups, C₂-C₂₀ alkenylene groups, C₂-C₂₀ alkynylene groups, C₃-C₂₀ cycloalkylene groups, C₅-C₂₀ cycloalkenylene groups, C₈-C₂₀ cycloalkynylene groups, C₇-C₂₀ alkylarylene groups, C₇-C₂₀ arylalkylene groups, C₈-C₂₀ arylalkenylene groups and C₉-C₂₀ arylalkynylene groups, the alkylene groups, alkenylene groups, alkynylene groups, cycloalkylene groups, cycloalkenylene groups, cycloalkynylene groups, alkylarylene groups, arylalkylene groups, arylalkenylene groups and arylalkynylene groups being optionally substituted and optionally interrupted by one or more heteroatoms selected from the group of O, S and NR²⁰, wherein R²° is independently selected from the group consisting of hydrogen, C₁-C₂₄ alkyl groups, C₂-C₂₄ alkenyl groups, C₂-C₂₄ alkynyl groups and C₃-C₂₄ cycloalkyl groups, the alkyl groups, alkenyl groups, alkynyl groups and cycloalkyl groups being optionally substituted.

In these preferred embodiments it is further preferred that the alkylene groups, alkenylene groups, alkynylene groups, cycloalkylene groups, cycloalkenylene groups, cycloalkynylene groups, alkylarylene groups, arylalkylene groups, arylalkenylene groups and arylalkynylene groups are unsubstituted and optionally interrupted by one or more heteroatoms selected from the group of O, S and NR²⁰, preferably O, wherein R²° is independently selected from the group consisting of hydrogen and C₁-C₄ alkyl groups, preferably hydrogen or methyl.

Most preferably, spacer moieties Sp¹ and Sp², if present, are independently selected from the group consisting of linear or branched C₁-C₂₀ alkylene groups, the alkylene groups being optionally substituted and optionally interrupted by one or more heteroatoms selected from the group of O, S and NR²⁰, wherein R²° is independently selected from the group consisting of hydrogen, C₁-C₂₄ alkyl groups, C₂-C₂₄ alkenyl groups, C₂-C₂₄ alkynyl groups and C₃-C₂₄ cycloalkyl groups, the alkyl groups, alkenyl groups, alkynyl groups and cycloalkyl groups being optionally substituted. In this embodiment, it is further preferred that the alkylene groups are unsubstituted and optionally interrupted by one or more heteroatoms selected from the group of O, S and NR²⁰, preferably O and/or S—S, wherein R²° is independently selected from the group consisting of hydrogen and C₁-Ca alkyl groups, preferably hydrogen or methyl.

Another class of suitable linkers comprises cleavable linkers. Cleavable linkers are well known in the art. For example Shabat et al., Soft Matter 2012, 6, 1073, incorporated by reference herein, discloses cleavable linkers comprising self-immolative moieties that are released upon a biological trigger, e.g. an enzymatic cleavage or an oxidation event. Some examples of suitable cleavable linkers are peptide-linkers that are cleaved upon specific recognition by a protease, e.g. cathepsin, plasmin or metalloproteases, or glycoside-based linkers that are cleaved upon specific recognition by a glycosidase, e.g. glucoronidase, or nitroaromatics that are reduced in oxygen-poor, hypoxic areas.

Linker L may further contain a peptide spacer as known in the art, preferably a dipeptide or tripeptide spacer as known in the art, preferably a dipeptide spacer. Although any dipeptide or tripeptide spacer may be used, preferably the peptide spacer is selected from Val-Cit, Val-Ala, Val-Lys, Val-Arg, AcLys-Val-Cit, AcLys-Val-Ala, Phe-Cit, Phe-Ala, Phe-Lys, Phe-Arg, Ala-Lys, Leu-Cit,Ile-Cit, Trp-Cit, Ala-Ala-Asn, Ala-Asn, more preferably Val-Cit, Val-Ala, Val-Lys, Phe-Cit, Phe-Ala, Phe-Lys, Ala-Ala-Asn, more preferably Val-Cit, Val-Ala, Ala-Ala-Asn. In one embodiment, the peptide spacer is Val-Cit. In one embodiment, the peptide spacer is Val-Ala. The peptide spacer may also be attached to the payload, wherein the amino end of the peptide spacer is conveniently used as amine group in the method according to the first aspect of the invention.

In a preferred embodiment, the peptide spacer is represented by general structure (L3):

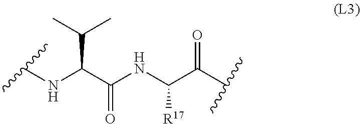

Herein, R¹⁷ =CH₃ (Val) or CH₂CH₂CH₂NHC(O)NH2 (Cit). The wavy lines indicate the connection to the remainder of the molecule, preferably the peptide spacer according to structure (L3) is connected to Q via NH and to D via C(O).

Linker L may further contain a self-cleavable spacer, also referred to as self-immolative spacer. The self-cleavable spacer may also be attached to the payload. Preferably, the self-cleavable spacer is para-aminobenzyloxycarbonyl (PABC) derivative, more preferably a PABC derivative according to structure (L4).

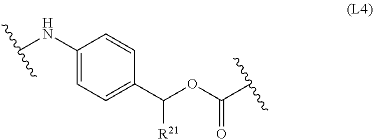

Herein, the wavy lines indicate the connection to the remainder of the molecule. Typically, the PABC derivative is connected via NH to Q, typically via a spacer, and via OC(O) to D, typically via a spacer.

R²¹ is H, R²² or C(O)R²², wherein R²² is C₁-C₂₄ (hetero)alkyl groups, C₃-C₁₀ (hetero)cycloalkyl groups, C₂-C₁₀ (hetero)aryl groups, C₃-C₁₀ alkyl(hetero)aryl groups and C₃-C₁₀ (hetero)arylalkyl groups, which optionally substituted and optionally interrupted by one or more heteroatoms selected from O, S and NR²³ wherein R²³ is independently selected from the group consisting of hydrogen and C₁-C₄ alkyl groups. Preferably, R²² is C₃-C₁₀ (hetero)cycloalkyl or polyalkylene glycol. The polyalkylene glycol is preferably a polyethylene glycol or a polypropylene glycol, more preferably —(CH₂CH₂O)ₛH or —(CH₂CH₂CH₂O)sH. The polyalkylene glycol is most preferably a polyethylene glycol, preferably —(CH₂CH₂O)sH, wherein s is an integer in the range 1-10, preferably 1-5, most preferably s=1, 2, 3 or 4. More preferably, R²¹ is H or C(O)R²², wherein R²²=4-methyl-piperazine or morpholine. Most preferably, R²¹ is H.

### Payload D

Linker L connects cyclic alkyne or alkene Q with payload D. Payload molecules are well-known in the art, especially in the field of antibody-drug conjugates, as the moiety that is covalently attached to the antibody and that is released therefrom upon uptake of the conjugate and/or cleavage of the linker. In a preferred embodiment, the payload is selected from the group consisting of an active substance, a reporter molecule, a polymer, a solid surface, a hydrogel, a nanoparticle, a microparticle and a biomolecule. Especially preferred payloads are active substances and reporter molecules, in particular active substances.

The term “active substance” herein relates to a pharmacological and/or biological substance, i.e. a substance that is biologically and/or pharmaceutically active, for example a drug, a prodrug, a cytotoxin, a diagnostic agent, a protein, a peptide, a polypeptide, a peptide tag, an amino acid, a glycan, a lipid, a vitamin, a steroid, a nucleotide, a nucleoside, a polynucleotide, RNA or DNA. Examples of peptide tags include cell-penetrating peptides like human lactoferrin or polyarginine. An example of a glycan is oligomannose. An example of an amino acid is lysine.

When the payload is an active substance, the active substance is preferably selected from the group consisting of drugs and prodrugs. More preferably, the active substance is selected from the group consisting of pharmaceutically active compounds, in particular low to medium molecular weight compounds (e.g. about 200 to about 2500 Da, preferably about 300 to about 1750 Da). In a further preferred embodiment, the active substance is selected from the group consisting of cytotoxins, antiviral agents, antibacterial agents, peptides and oligonucleotides. Examples of cytotoxins include colchicine, vinca alkaloids, anthracyclines, camptothecins, doxorubicin, daunorubicin, taxanes, calicheamycins, tubulysins, irinotecans, an inhibitory peptide, amanitin, deBouganin, duocarmycins, maytansines, auristatins, enediynes, pyrrolobenzodiazepines (PBDs) or indolinobenzodiazepine dimers (IGN) or PNU159,682 and derivatives thereof. Preferred payloads are selected from MMAE, MMAF, exatecan, SN-38, DXd, maytansinoids, calicheamicin, PNU159,685 and PBD dimers. Especially preferred payloads are PBD, SN38, MMAE, exatecan or DXd. In one embodiment, the payload is MMAE. In one embodiment, the payload is exatecan or DXd. In one embodiment, the payload is SN-38. In one embodiment, the payload is MMAE. In one embodiment, the payload is a PDB dimer.

The term “reporter molecule” herein refers to a molecule whose presence is readily detected, for example a diagnostic agent, a dye, a fluorophore, a radioactive isotope label, a contrast agent, a magnetic resonance imaging agent or a mass label.

A wide variety of fluorophores, also referred to as fluorescent probes, is known to a person skilled in the art. Several fluorophores are described in more detail in e.g. G. T. Hermanson, “Bioconjugate Techniques”, Elsevier, 3^{rd} Ed. 2013, Chapter 10: “Fluorescent probes”, p. 395 - 463, incorporated by reference. Examples of a fluorophore include all kinds of Alexa Fluor (e.g. Alexa Fluor 555), cyanine dyes (e.g. Cy3 or Cy5) and cyanine dye derivatives, coumarin derivatives, fluorescein and fluorescein derivatives, rhodamine and rhodamine derivatives, boron dipyrromethene derivatives, pyrene derivatives, naphthalimide derivatives, phycobiliprotein derivatives (e.g. allophycocyanin), chromomycin, lanthanide chelates and quantum dot nanocrystals.

Examples of a radioactive isotope label include ^{99m}Tc, 111In ^{114m}In, ¹¹⁵In, ¹⁸_{F,} ¹⁴C, ⁶⁴Cu, ¹³¹I, ¹²⁵I, ¹²³I, ²¹²Bi, ⁸⁸Y, ⁹⁰Y, ⁶⁷Cu, ¹⁸⁶Rh, ⁶⁶Ga, ⁶⁷Ga and ¹⁰B which is optionally connected via a chelating moiety such as e.g. DTPA (diethylenetriaminepentaacetic anhydride), DOTA (1,4,7,10-tetraazacyclododecane-N,N;N″,N′″-tetraacetic acid), NOTA (1,4,7-triazacyclononane N,N′,N″-triacetic acid), TETA (1,4,8,11-tetraazacyclotetradecane-N,N;N″,Ar-tetraacetic acid), DTTA (N¹-(p-isothiocyanatobenzyl)-diethylenetriamine-N¹, N²,N³,N³-tetraacetic acid), deferoxamine or DFA (N′-(5-5-[[4-[[5-(acetylhydroxyamino)pentyl]amino]-1,4-dioxobutyl]hydroxyamino]pentyl-N-(5-aminopentyl)-N-hydroxybutanediamide) or HYNIC (hydrazinonicotinamide). Isotopic labelling techniques are known to a person skilled in the art, and are described in more detail in e.g. G.T. Hermanson, “Bioconjugate Techniques”, Elsevier, 3¹c¹ Ed. 2013, Chapter 12: “Isotopic labelling techniques”, p. 507 - 534, incorporated by reference.

Polymers suitable for use as a payload D in the compound according to the invention are known to a person skilled in the art, and several examples are described in more detail in e.g. G.T.

Hermanson, “Bioconjugate Techniques”, Elsevier, 3¹d Ed. 2013, Chapter 18: “PEGylation and synthetic polymer modification”, p. 787 - 838, incorporated by reference. When payload D is a polymer, payload D is preferably independently selected from the group consisting of a poly(ethyleneglycol) (PEG), a polyethylene oxide (PEO), a polypropylene glycol (PPG), a polypropylene oxide (PPO), a 1 ,x-diaminoalkane polymer (wherein xis the number of carbon atoms in the alkane, and preferably x is an integer in the range of 2 to 200, preferably 2 to 10), a (poly)ethylene glycol diamine (e.g. 1,8-diamino-3,6-dioxaoctane and equivalents comprising longer ethylene glycol chains), a polysaccharide (e.g. dextran), a poly(amino acid) (e.g. a poly(L-lysine)) and a poly(vinyl alcohol).

Solid surfaces suitable for use as a payload D are known to a person skilled in the art. A solid surface is for example a functional surface (e.g. a surface of a nanomaterial, a carbon nanotube, a fullerene or a virus capsid), a metal surface (e.g. a titanium, gold, silver, copper, nickel, tin, rhodium or zinc surface), a metal alloy surface (wherein the alloy is from e.g. aluminum, bismuth, chromium, cobalt, copper, gallium, gold, indium, iron, lead, magnesium, mercury, nickel, potassium, plutonium, rhodium, scandium, silver, sodium, titanium, tin, uranium, zinc and/or zirconium), a polymer surface (wherein the polymer is e.g. polystyrene, polyvinylchloride, polyethylene, polypropylene, poly(dimethylsiloxane) or polymethylmethacrylate, polyacrylamide), a glass surface, a silicone surface, a chromatography support surface (wherein the chromatography support is e.g. a silica support, an agarose support, a cellulose support or an alumina support), etc. When payload D is a solid surface, it is preferred that D is independently selected from the group consisting of a functional surface or a polymer surface.

Hydrogels are known to the person skilled in the art. Hydrogels are water-swollen networks, formed by cross-links between the polymeric constituents. See for example A. S. Hoffman, Adv. Drug Delivery Rev. 2012, 64, 18, incorporated by reference. When the payload is a hydrogel, it is preferred that the hydrogel is composed of poly(ethylene)glycol (PEG) as the polymeric basis.

Micro- and nanoparticles suitable for use as a payload D are known to a person skilled in the art. A variety of suitable micro- and nanoparticles is described in e.g. G.T. Hermanson, “Bioconjugate Techniques”, Elsevier, 3¹c¹ Ed. 2013, Chapter 14: “Microparticles and nanoparticles”, p. 549 - 587, incorporated by reference. The micro- or nanoparticles may be of any shape, e.g. spheres, rods, tubes, cubes, triangles and cones. Preferably, the micro- or nanoparticles are of a spherical shape. The chemical composition of the micro- and nanoparticles may vary. When payload D is a micro- or a nanoparticle, the micro- or nanoparticle is for example a polymeric micro- or nanoparticle, a silica micro- or nanoparticle or a gold micro- or nanoparticle. When the particle is a polymeric micro- or nanoparticle, the polymer is preferably polystyrene or a copolymer of styrene (e.g. a copolymer of styrene and divinylbenzene, butadiene, acrylate and/or vinyltoluene), polymethylmethacrylate (PMMA), polyvinyltoluene, poly(hydroxyethyl methacrylate (pHEMA) or poly(ethylene glycol dimethacrylate/2-hydroxyethylmetacrylae) [poly(EDGMA/HEMA)]. Optionally, the surface of the micro- or nanoparticles is modified, e.g. with detergents, by graft polymerization of secondary polymers or by covalent attachment of another polymer or of spacer moieties, etc.

Payload D may also be a biomolecule. Biomolecules, and preferred embodiments thereof, are described in more detail below. When payload D is a biomolecule, it is preferred that the biomolecule is selected from the group consisting of proteins (including glycoproteins such as antibodies), polypeptides, peptides, glycans, lipids, nucleic acids, oligonucleotides, polysaccharides, oligosaccharides, enzymes, hormones, amino acids and monosaccharides.

In the context of the present invention, cytotoxic payloads are especially preferred. Thus, D is preferably, a cytotoxin, more preferably selected from the group consisting of colchicine, vinca alkaloids, anthracyclines, camptothecins, doxorubicin, daunorubicin, taxanes, calicheamycins, tubulysins, irinotecans, an inhibitory peptide, amanitins, amatoxins, deBouganin, duocarmycins, epothilones, mytomycins, combretastatins, maytansines, auristatins, enediynes, pyrrolobenzodiazepines (PBDs) or indolinobenzodiazepine dimers (IGN) or PNU159,682. In an especially preferred embodiment, D is MMAE or exatecan.

The cyclic alkyne or alkene compound of structure Q—L—D (2) is preferably represented by a structure selected from the group consisting of (2a)-(2t):

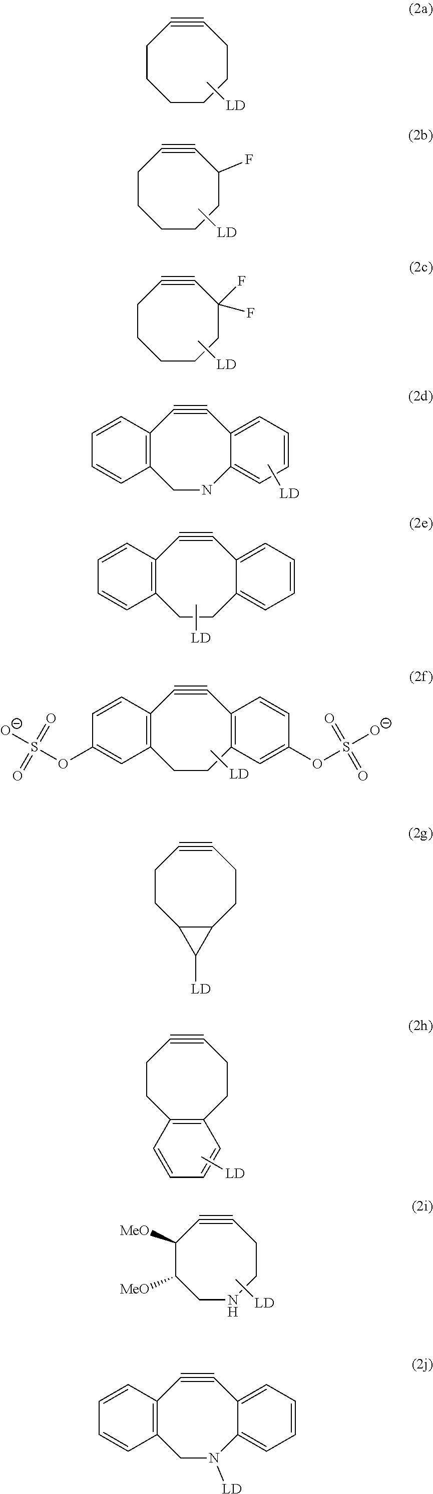

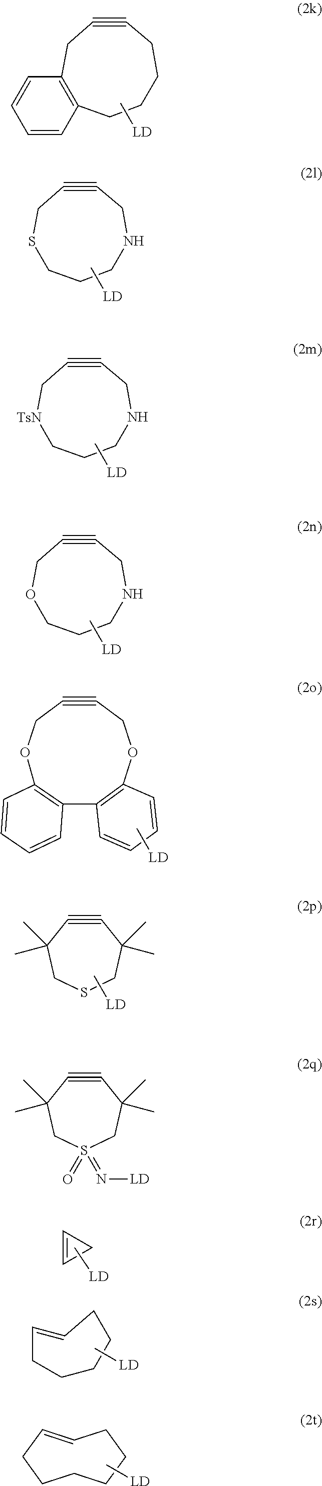

In a further preferred embodiment, the cyclic alkyne or alkene compound of structure Q—L—D (2) is represented by a structure selected from the group consisting of (2aa)-(2ba):

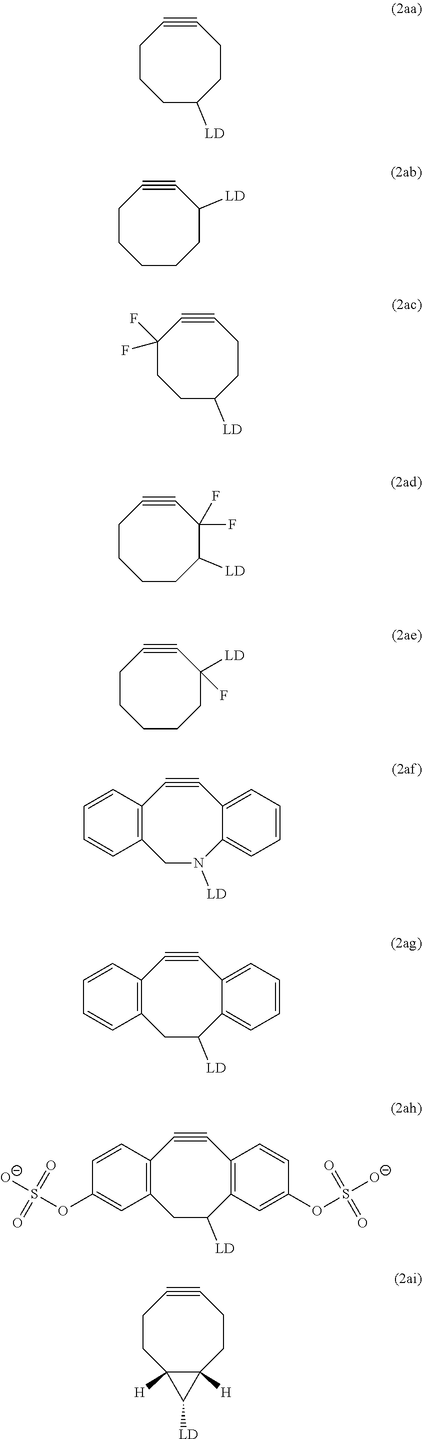

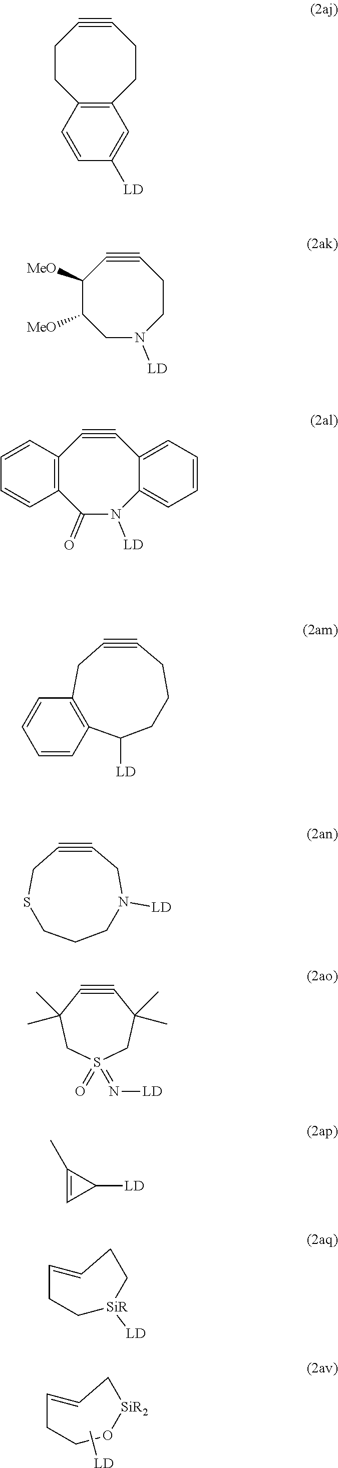

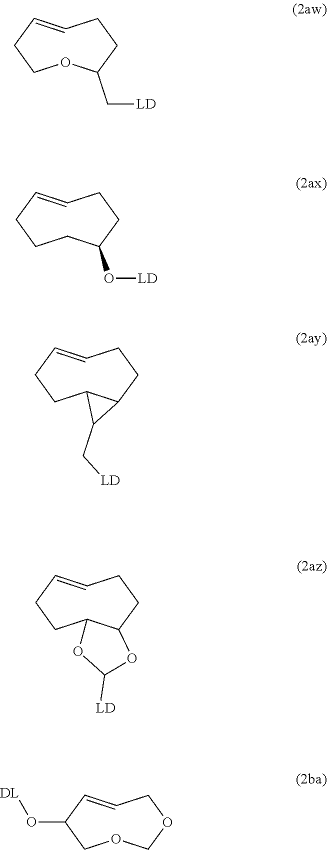

The definition and preferred embodiments of L and D, as provided above, equally apply to the compounds of structure (2a)-(2t) and (2aa)-(2ba). The R group(s) on Si in (2aq) and (2av) are typically alkyl or aryl, preferably C₁-C₆ alkyl. Especially preferred cyclic alkyne or alkene compounds of structure Q—L—D (2) in the context of the present invention are depicted in FIGS. 7A-7F, even more preferably in FIGS. 7A-7C.

### EXAMPLES

The invention is illustrated by the following examples.

### General Procedure for Analytical RP-HPLC

Prior to RP-HPLC analysis, IgG (10 μL, 1 mg/mL in PBS pH 7.4) was added to 12.5 mM DTT, 100 mM TrisHCl pH 8.0 (40 μL) and incubated for 15 minutes at 37° C. The reaction was quenched by adding 49% acetonitrile, 49% water, 2% formic acid (50 μL). RP-HPLC analysis was performed on an Agilent 1100 series (Hewlett Packard). The sample (10 μL) was injected with 0.5 mL/min onto Bioresolve RP mAb 2.'1150 mm 2.7 pm (Waters) with a column temperature of 70° C. A linear gradient was applied in 16.8 minutes from 30 to 54% acetonitrile in 0.1% TFA and water.

### General Procedure for Analytical SEC

HPLC-SEC analysis was performed on an Agilent 1100 series (Hewlett Packard) using an Xbridge BEH200A (3.5 pM, 7.8x300 mm, PN 186007640 Waters) column. The sample was diluted to 1 mg/mL in PBS and measured with 0.86 mL/min isocratic method (0.1 M sodium phosphate buffer pH 6.9 (NaHPO_{4/}Na2PO₄) containing 10% isopropanol) for 16 minutes.

### General Procedure for Mass Spectral Analysis of Monoclonal Antibodies

Prior to mass spectral analysis, IgG was treated with IdeS, which allows analysis of the Fc/2 fragment. For analysis of the Fc/2 fragment, a solution of 20 pg (modified) IgG was incubated for 1 hour at 37° C. with IdeS/Fabricator™ (1.25 U/μL) in PBS pH 6.6 in a total volume of 10 μL. Samples were diluted to 80 μL followed by analysis electrospray ionization time-of-flight (ESI-TOF) on a JEOL AccuTOF. Deconvoluted spectra were obtained using Magtran software.

### General Procedure for Analytical RP-UPLC

Prior to RP-UPLC analysis, IgG (10 μL, 1 mg/mL in PBS pH 7.4) was added to 12.5 mM DTT, 100 mM Tris.HCI pH 8.0 (40 μL) and incubated for 15 minutes at 37° C. The reaction was quenched by adding 49% acetonitrile, 49% water, 2% formic acid (50 μL). RP-UPLC analysis was performed on a Waters Acquity UPLC-SQD. The sample (5 μL) was injected with 0.4 mL/min onto Bioresolve RP mAb 2.1×150 mm 2.7 pm (Waters) with a column temperature of 70° C. A linear gradient was applied in 9 minutes from 30 to 54% acetonitrile in 0.1% TFA and water.

General procedure for analytical RP-UPLC with prior quench with 1-azidomethylpyrene

Prior to RP-UPLC analysis 5 μL of a 1 mM solution of 1-azidomethylpyrene (TCI Europe) in DMF was added to the solution of IgG (50 μL, 1 mg/mL in PBS pH 7.4) and the mixture was incubated for 4 hours at RT. The mixture was spin-filtered to PBS and RP-UPLC analysis intact sample was performed on a Waters Acquity UPLC-SQD. The sample (5 μL) was injected with 0.4 mL/min onto Bioresolve RP mAb 2.1×150 mm 2.7 pm (Waters) with a column temperature of 70 ° C. A linear gradient was applied in 9 minutes from 30 to 54% acetonitrile in 0.1% TFA and water.

### Example 1

### Synthetic Preparation

Compounds X1 and X2 were prepared according to Verkade et al., Antibodies 2018, 12, doi:10.3390/antib7010012. Compounds X5A, X9 and X10 were prepared according to WO 2019/110725 (respectively compounds 150, 140 and 157). Compound X6 was prepared according to WO 2018/146189 (compound 4). Compound X8 was prepared according to WO 2017/137457 (compound 56). Compounds X11 and X12 were prepared according to WO 2021/144313 (respectively compounds 137 and 304).

### Example 2

### Enzymatic Remodeling of Rituximab to Rituximab-(6-N3-GaINAc)₂

Rituximab (15 mg/mL) was incubated with EndoSH (1% w/w), as described in PCT/EP2017/052792 (WO 2017/137459), His-TnGaINAcT, described in PCT/EP2016/059194 (WO 2016/170186) (5% w/w) and UDP 6-N3-GaINAc (25 eq compared to IgG), prepared according to PCT/EP2016/059194 (WO 2016/170186) in TBS containing 10 mM MnCl2 for 16 hours at 30° C. Next, the functionalized IgG was purified using a HiTrap MabSelect Sure 5 mL column. After loading of the reaction mixture the column was washed with TBS +0.2% Triton and TBS. The IgG was eluted with 0.1 M glycine-HCI pH 2.7 and neutralized with 1 M Tris-HCI pH 8.8. After three times dialysis to PBS, the IgG was concentrated to 15-20 mg/mL using a Vivaspin Turbo 15 ultrafiltration unit (Sartorius).

### Example 3

### Enzymatic Remodeling of Trastuzumab to Trastuzumab-(6-N3-GaINAc)₂

Trastuzumab (15 mg/mL) was incubated with EndoSH (1% w/w), as described in PCT/EP2017/052792 (WO 2017/137459), His-TnGaINAcT, described in PCT/EP2016/059194 (WO 2016/170186) (5% w/w) and UDP 6-N3-GaINAc (25 eq compared to IgG), prepared according to PCT/EP2016/059194 (WO 2016/170186) in TBS containing 10 mM MnCl2 for 16 hours at 30° C.

Next, the functionalized IgG was purified using a HiTrap MabSelect Sure 5 mL column. After loading of the reaction mixture the column was washed with TBS +0.2% Triton and TBS. The IgG was eluted with 0.1 M glycine-HCI pH 2.7 and neutralized with 1 M Tris-HCI pH 8.8. After three times dialysis to PBS, the IgG was concentrated to 15-20 mg/mL using a Vivaspin Turbo 15 ultrafiltration unit (Sartorius).

### Example 4

### Screening different Surfactants at 10 mg/mL Antibody Concentration

Rituximab-(6-N3-GaINAc)₂ (10 mg/mL, 0.2 mg) was incubated overnight with compound X1 or compound X2 (0.125-0.2 mM (2-3 equiv.) with 10% DMF. Optionally, sodium deoxycholate (11 mM), sodium decanoate (37.5 mM) or CHAPS (12 mM) were added. After 16 h, reactions were analyzed with RP-HPLC analysis (after DTT reduction) to determine the drug:antibody ratio (DAR). Results are depicted in FIG. 6.

### Example 5

### Comparison at 15 mg/mL and 10% DMF with compound X1 (structure in FIG. 7A)

Rituximab-(6-N3-GaINAc)₂ (15 mg/mL, 0.2 mg) was incubated overnight with X1 (0.26 mM, 3 equiv.) with 10% DMF and optionally sodium decanoate (37.5 mM) or sodium deoxycholate (11 mM) were added. After 16 h, reactions were analyzed with RP-HPLC analysis (after reduction) to determine the DAR. Results are depicted in the Table below.

| Additive | DAR |
|---|---|
| None | 3.45 |
| Sodium decanoate | 3.68 |
| Sodium deoxycholate | 3.71 |

### Example 6

### Comparison at 15 mg/mL and 10% DMF with compound X2 (structure in FIG. 7A)

Rituximab-(6-N3-GaINAc)₂ (15 mg/mL, 0.2 mg) was incubated overnight with X2 (0.3 mM, 3 equiv.) with 10% DMF and optionally sodium deoxycholate (11 mM) was added. After 16 h, reactions were analyzed with RP-HPLC analysis (after reduction) to determine the DAR. Results are depicted in the Table below.

| Additive | DAR |
|---|---|
| None | 2.12 |
| Sodium deoxycholate | 3.39 |

### Example 7

### Coniuciation with X2 in Propylene Glycol (PG)

Trastzumab-(6-N3-GaINAc)₂ (10 mg/mL, 0.2 mg) was incubated overnight with X2 0.4 mM (6 equiv.) or 0.33 mM (5 equiv.) with 30% PG and either no additive or 11 mM sodium deoxycholate. After 16 h, reactions were analyzed with RP-HPLC analysis (after DTT reduction) to determine the DAR.

| Conditions | DAR |
|---|---|
| No additive, 30% PG, 6 equiv. | 2.36 |
| Sodium deoxycholate, 30% PG, 6 equiv. | 3.61 |
| Sodium deoxycholate, 30% PG, 5 equiv. | 3.64 |

### Example 8

### Comparison at 15-10 mci/mL and 5% DMF with compound X1

Trastuzumab-(6-N3-GaINAc)₂ (10-15 mg/mL, 0.2 mg) was incubated overnight with X1 (0.2-0.3 mM , 3 equiv) with 5% DMF and sodium deoxycholate (22 mM) were added. After 16 h, reactions was analyzed with RP-HPLC analysis (after reduction) to determine the DAR. Results are depicted in the Table below.

| concentration | DAR |
|---|---|
| 10 mg/mL | 3.00 |
| 15 mg/mL | 3.62 |

### Example 9

### Coniuciation with X2 in Propylene Glycol (PG)

Trastzumab-(6-N3-GaINAc)₂ (10 mg/mL, 0.2 mg) was incubated overnight with X2 0.33 mM (5 equiv) with 20-25-30% PG and 11-22 mM sodium deoxycholate. After 16 h, reactions were analyzed with RP-HPLC analysis (after DTT reduction) to determine the DAR.

| Conditions | DAR |
|---|---|
| 11 mM sodium deoxycholate, 20% PG | 2.36 |
| 11 mM sodium deoxycholate, 25% PG | 2.72 |
| 11 mM sodium deoxycholate, 30% PG | 3.65 |
| 22 mM sodium deoxycholate, 20% PG | 3.65 |
| 22 mM sodium deoxycholate, 25% PG | 3.65 |
| 22 mM sodium deoxycholate, 30% PG | 3.67 |

### Example 10

### Comparison at 15 mci/mL and 10% DMF with compound X5A

Trastuzumab-(6-N3-GaINAc)₂ (15 mg/mL, 0.3 mg) was incubated overnight with X5A (2 equiv vs. antibody) with 10% DMF and optionally sodium deoxycholate (11 mM) was added. After 16 h, reactions were analyzed with RP-HPLC analysis (after DTT reduction) to determine the DAR. Results are depicted in the Table below. A clear improvement in DAR is noted in case sodium deoxycholate is used during conjugation.

| Quantity of X5A | Additive | DAR |
|---|---|---|
| 2 equiv | None | 1.58 |
| | Sodium deoxycholate (11 mM) | 1.80 |

### Example 11

### Comparison at 10 mci/mL and 10% DMF with compound X6

Trastuzumab-(6-N3-GaINAc)₂ (10 mg/mL, 0.3 mg) was incubated overnight with X6 (2 equiv vs. antibody) with 10% DMF and optionally sodium deoxycholate (11 mM) was added. After 16 h, reactions were analyzed with RP-UPLC analysis (after DTT reduction) to determine the DAR. Results are depicted in the Table below. A clear improvement in DAR is noted in case sodium deoxycholate is used during conjugation.

| Quantity of X6 | Additive | DAR |
|---|---|---|
| 2 equiv | None | 1.17 |
| | Sodium deoxycholate (11 mM) | 1.37 |

### Example 12

### Comparison at 15 mci/mL and 10% DMF with compound X8

Trastuzumab-(6-N3-GaINAc)₂ (15 mg/mL, 0.3 mg) was incubated overnight with X8 (2 or 3 equiv vs. antibody) with 10% DMF and optionally CHAPS (12 mM), sodium deoxycholate (11 mM) or sodium decanoate (37.5 mM) was added. After 16 h, reactions were analyzed with RP-UPLC analysis (after DTT reduction) to determine the drug-to-antibody ratio (DAR). Results are depicted in the Table below. A great improvement in DAR is noted in case sodium deoxycholate or sodium decanoate are used during conjugation.

| Quantity of X8 | Additive | DAR |
|---|---|---|
| 2 equiv | None | 0.11 |
| | CHAPS (12 mM) | 0.27 |
| | Sodium deoxycholate (11 mM) | 2.04 |
| | Sodium decanoate (37.5 mM) | 2.77 |
| 3 equiv | None | 0.11 |
| | CHAPS (12 mM) | 0.23 |
| | Sodium deoxycholate (11 mM) | 2.32 |
| | Sodium decanoate (37.5 mM) | 3.62 |

### Example 13

### Comparison at 15 mci/mL and 10% DMF with compound X9

Trastuzumab-(6-N3-GaINAc)₂ (15 mg/mL, 0.3 mg) was incubated overnight with X9 (2 or 3 equiv vs. antibody) with 10% DMF and optionally sodium deoxycholate (11 mM) was added. After 16 h, reactions were analyzed with RP-UPLC analysis (after DTT reduction) to determine the DAR. Results are depicted in the Table below. A great improvement in DAR is noted in case sodium deoxycholate is used during conjugation.

| Quantity of X9 | Additive | DAR |
|---|---|---|
| 2 equiv | None | 0.02 |
| | Sodium deoxycholate (11 mM) | 0.93 |
| 3 equiv | None | 0.00 |
| | Sodium deoxycholate (11 mM) | 0.96 |

### Example 14

### Comparison at 15 mci/mL and 10% DMF with compound X10

Trastuzumab-(6-N3-GaINAc)₂ (15 mg/mL, 0.3 mg) was incubated overnight with X10 (2 or 3 equiv vs. antibody) with 10% DMF and optionally sodium deoxycholate (11 mM) was added. After 16 h, reactions were analyzed with RP-UPLC analysis (after DTT reduction) to determine the DAR.

Results are depicted in the Table below. A clear improvement in DAR is noted in case sodium deoxycholate is used during conjugation.

| Quantity of X10 | Additive | DAR |
|---|---|---|
| 2 equiv | None | 0.45 |
| | Sodium deoxycholate (11 mM) | 0.72 |
| 3 equiv | None | 0.44 |
| | Sodium deoxycholate (11 mM) | 1.17 |

### Example 15

### Comparison at 5 mci/mL and 10% DMF with compound X11

Trastuzumab-(6-N3-GaINAc)₂ (5 mg/mL, 0.3 mg) was incubated overnight with X11 (1.5 or 2.5 equiv vs. antibody) with 10% DMF and optionally sodium deoxycholate (11 mM) was added. After 16 h, reactions were analyzed with RP-UPLC analysis (after DTT reduction) to determine the

DAR, and RP-UPLC analysis (intact samples) to determine relative amounts of “DARO” (unconjugated antibody), “DAR1” (closed DAR1 conjugate, where two click reactions have occurred between both azido moieties of a single antibody and both BCN moieties of a single compound X11; and open DAR1 conjugates, where only click reaction has occurred between an azido moiety and a BCN moiety) and “DAR2” conjugates (where both azido moieties of a single antibody have reacted with two BCN moieties of different compounds X11). Results are depicted in the Table below. A clear improvement in %DAR1 is noted in case sodium deoxycholate is used during conjugation.

| Quantity | | | | |
|---|---|---|---|---|
| of X11 | Additive | DAR0 | DAR1 * | DAR2 |
| 1.5 equiv | None | 60.6% | 39.4% | 0.0% |
| | Sodium deoxycholate (11 mM) | 16.2% | 82.6% | 1.2% |
| 2.5 equiv | None | 62.2% | 37.8% | 0.0% |
| | Sodium deoxycholate (11 mM) | 2.2% | 93.4% | 4.4% |

* Total of “open” and “closed” DAR1 conjugates.

### Example 16

### Comparison at 5 mg/mL and 10% DMF with compound X12

Trastuzumab-(6-N3-GaINAc)₂ (5 mg/mL, 0.3 mg) was incubated overnight with X12 (1.5 or 2.5 equiv vs. antibody) with 10% DMF and optionally sodium deoxycholate (11 mM) was added. After 16 h, reactions were analyzed with RP-UPLC analysis (intact samples after quenching with 1-azidomethylpyrene) to determine relative amounts of “DARO” (unconjugated antibody), “DAR1” (closed DAR1 conjugate, where two click reactions have occurred between both azido moieties of a single antibody and both BCN moieties of a single compound X12) and “other” conjugates (open DAR1 conjugates, where only click reaction has occurred between an azido moiety and a BCN moiety; and DAR2 conjugates where both azido moieties of a single antibody have reacted with two BCN moieties of different compounds X12). Results are depicted in the Table below. A clear improvement in %DAR1 is noted in case sodium deoxycholate is used during conjugation.

| Quantity | | | | |
|---|---|---|---|---|
| of X12 | Additive | DAR0 | DAR1 * | other |
| 1.5 equiv | None | 40.7% | 48.7% | 10.6% |
| | Sodium deoxycholate (11 mM) | 36.2% | 55.7% | 8.1% |
| 2.5 equiv | None | 28.2% | 48.9% | 22.9% |
| | Sodium deoxycholate (11 mM) | 22.3% | 62.2% | 15.5% |

* Only “closed” DAR1 conjugates; “open” DAR1 conjugates are part of “other” in the final column.

### Example 17

### Coniuciation of Trastuzumab-(6-N3-GaINAc)₂ with Compound X1 in the Absence of Sodium Deoxycholate (comparative)

Trastuzumab-(6-N3-GaINAc)₂ (15 mg/mL, 7 mg) was incubated overnight with X1 (7 equiv) with 25% DMF. After 16 h, reactions were analyzed with RP-HPLC analysis (after DTT reduction) to determine the DAR (3.7). Subsequently, the reaction was diluted to 2.5 mL with TBS and subsequent concentrated to 1 mL using amicon 10 kDa spinfilters, and then purified on an AKTA Purifier-10 (GE Healthcare) with a Superdex200 Increase 10/300 GL (GE Healthcare) column to yield the conjugate in 80% yield.

### Example 18

### Coniuciation of Trastuzumab-(6-N3-GaINAc)₂ with compound X1 in the presence of Sodium Deoxycholate

Trastuzumab-(6-N3-GaINAc)₂ (15 mg/mL, 10 mg) was incubated overnight with X1 (3 equiv) with 10% DMF and sodium deoxycholate (11 mM). After 16 h, reactions were analyzed with RP-HPLC analysis (after DTT reduction) to determine the DAR (3.7). Subsequently, the reaction was directly purified on an AKTA Purifier-10 (GE Healthcare) with a Superdex200 Increase 10/300 GL (GE Healthcare) column to yield the conjugate in 89% yield.

The results of examples 17 and 18 show that the presence of the surfactant provides a higher yield. Also, the downstream processing (work-up, purification) of the conjugate after the conjugation reaction is simplified, as the dialysis step with amicon 10 kDa spinfilters was not needed for the conjugation reaction in the presence of surfactant.

1. A method for the preparation of a bioconjugate of structure B—(Z—L—D),, comprising reacting:
(i) an alkyne or alkene compound of structure Q—L—D, wherein
(a) Q is a click probe comprising a cyclic alkyne moiety or a cyclic alkene moiety,
(b) L is a linker, and
(c) D is a payload; with
(ii) a molecule of structure B—(F)ₓ, wherein
(a) B is a biomolecule that is functionalized with x click probes F;
(b) F is a click probe capable of reacting with Q, and
(c) xis an integer in the range of 1-10,
in presence of a surfactant, to form a bioconjugate wherein the payload is covalently attached to the biomolecule via connecting group Z that is formed by a click reaction between Q and F.

2. The method according to claim 1, wherein the surfactant contains a negatively charged moiety.

3. The method according to claim 1, wherein the surfactant is selected from the group consisting of sodium decanoate, sodium dodecanoate, sodium lauryl sulfate (SDS), sodium deoxycholate.

4. The method according to claim 1, wherein the reaction is performed in a solvent system containing water and organic solvent in a ratio in the range of 50/50-100/0.

5. The method according to claim 4, wherein the reaction is performed in a solvent system containing water and organic solvent in a ratio in the range of 75/25-95/5.

6. The method according to claim 1, wherein the concentration of the molecule of structure is in the range of 1-100 mg/mL.

7. The method according to claim 6, wherein the concentration of the molecule of structure is in the range of 5-50 mg/mL.

8. The method according to claim 1, wherein the click probe Q comprises a cyclic alkyne moiety and click probe F is selected from the group consisting of azide, tetrazine, triazine, nitrone, nitrile oxide, nitrile imine, diazo compound, ortho-quinone, dioxothiophene and sydnone.

9. The method according to claim 1, wherein the click probe Q is selected from the group consisting of (Q22)-(Q36): 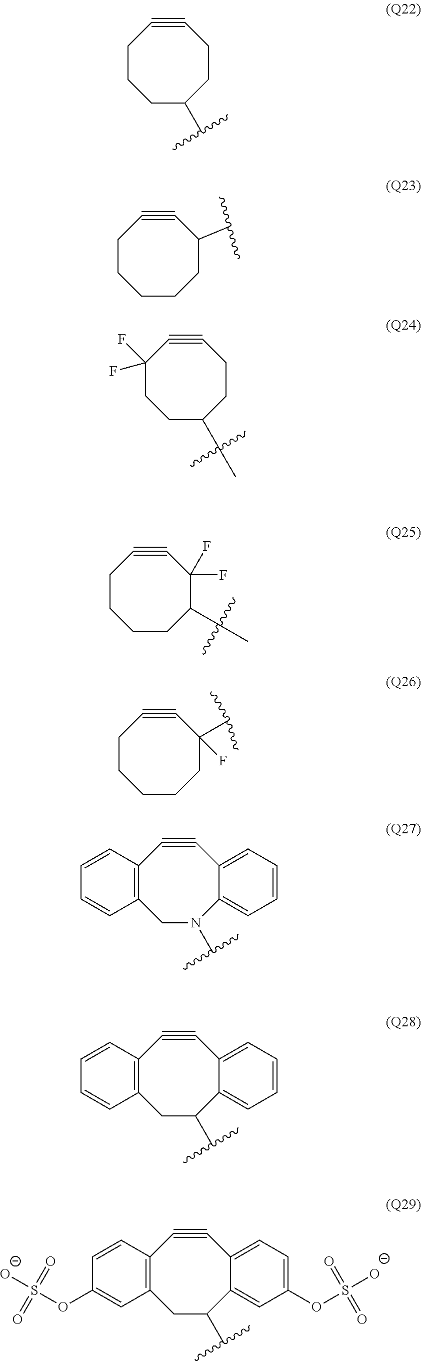 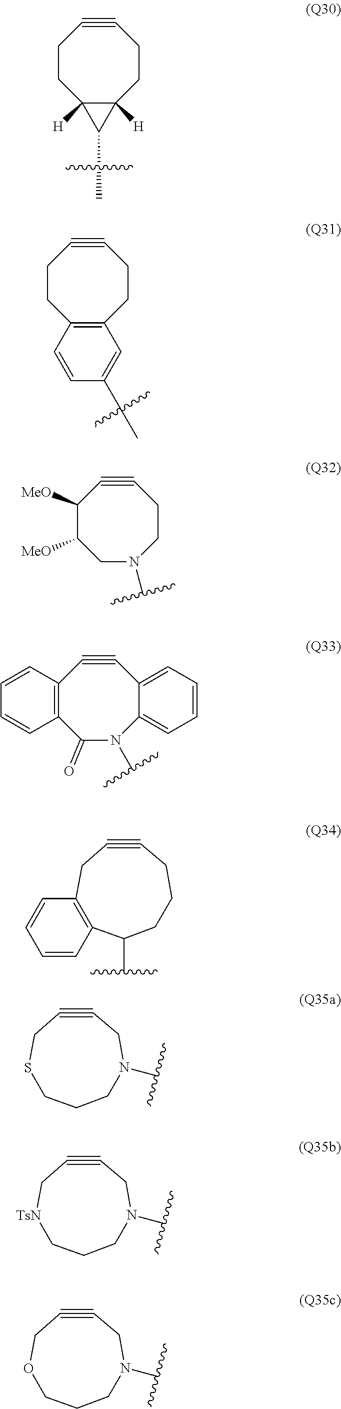 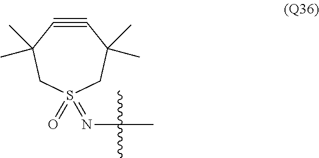
or wherein the (hetero)cycloalkynyl moiety Q is according to structure (Q37): 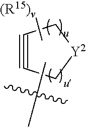
(Q37)
wherein:
R¹⁵ is independently selected from the group consisting of hydrogen, halogen, —OR¹⁶, —NO₂, —CN, —S(O)₂R¹⁶, —S(O)₃⁽⁻⁾, C₁-C₂₄ alkyl groups, C₆-C₂₄ (hetero)aryl groups, C₇-C₂₄ alkyl(hetero)aryl groups and C₇-C₂₄ (hetero)arylalkyl groups and wherein the alkyl groups, (hetero)aryl groups, alkyl(hetero)aryl groups and (hetero)arylalkyl groups are optionally substituted, wherein two substituents R¹⁵ may be linked together to form an optionally substituted annulated cycloalkyl or an optionally substituted annulated (hetero)arene substituent, and wherein R¹⁶ is independently selected from the group consisting of hydrogen, halogen, C₁-C₂₄ alkyl groups, C₆-C₂₄ (hetero)aryl groups, C₇-C₂₄ alkyl(hetero)aryl groups and C₇-C₂₄ (hetero)arylalkyl groups;
y² is C (R³¹)₂, O, S or NR³¹, wherein each R³¹ individually is R¹⁵ or —LD;
u is 0, 1, 2, 3, 4 or 5;
u′ is 0, 1, 2, 3, 4 or 5, wherein u+u′=4, 5, 6, 7 or 8;
v=an integer in the range 8-16;
or wherein the cyclooctynyl moiety Q is according to structure (Q38): 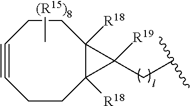
(Q38)
wherein
R¹⁵ is independently selected from the group consisting of hydrogen, halogen, —OR¹⁶, —NO₂, —CN, —S(O)₂R¹⁶, —S(O)₃⁽⁻⁾, C₁-C₂₄ alkyl groups, C₅-C₂₄ (hetero)aryl groups, C₇-C₂₄ alkyl(hetero)aryl groups and C₇-C₂₄ (hetero)arylalkyl groups and wherein the alkyl groups, (hetero)aryl groups, alkyl(hetero)aryl groups and (hetero)arylalkyl groups are optionally substituted, wherein two substituents R¹⁵ may be linked together to form an optionally substituted annulated cycloalkyl or an optionally substituted annulated (hetero)arene substituent, and wherein R¹⁶ is independently selected from the group consisting of hydrogen, halogen, C₁-C₂₄ alkyl groups, C₆-C₂₄ (hetero)aryl groups, C₇-C₂₄ alkyl(hetero)aryl groups and C₇-C₂₄ (hetero)arylalkyl groups;
R¹⁸ is independently selected from the group consisting of hydrogen, halogen, C₁-C₂₄ alkyl groups, C₆-C₂₄ (hetero)aryl groups, C₇-C₂₄ alkyl(hetero)aryl groups and C₇-C₂₄ (hetero)arylalkyl groups;
R¹⁹ is selected from the group consisting of hydrogen, —LD; halogen, C₁-C₂₄ alkyl groups, C₆-C₂₄ (hetero)aryl groups, C₇-C₂₄ alkyl(hetero)aryl groups and C₇-C₂₄ (hetero)arylalkyl groups, the alkyl groups optionally being interrupted by one of more hetero-atoms selected from the group consisting of O, N and S, wherein the alkyl groups, (hetero)aryl groups, alkyl(hetero)aryl groups and (hetero)arylalkyl groups are independently optionally substituted; and
I is an integer in the range 0 to 10;
or wherein the (hetero)cyclooctynyl moiety Q is according to structure (Q39): 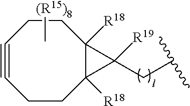
(Q39)
wherein
R¹⁵ is independently selected from the group consisting of hydrogen, halogen, —OR¹⁶, -NO₂, -CN, —S(O)₂R¹⁶, —S(O)₃⁽⁻⁾, C₁-C₂₄ alkyl groups, C₅-C₂₄ (hetero)aryl groups, C₇-C₂₄ alkyl(hetero)aryl groups and C₇-C₂₄ (hetero)arylalkyl groups and wherein the alkyl groups, (hetero)aryl groups, alkyl(hetero)aryl groups and (hetero)arylalkyl groups are optionally substituted, wherein two substituents R¹⁵ may be linked together to form an optionally substituted annulated cycloalkyl or an optionally substituted annulated (hetero)arene substituent, and wherein R¹⁶ is independently selected from the group consisting of hydrogen, halogen, C₁-C₂₄ alkyl groups, C₆-C₂₄ (hetero)aryl groups, C₇-C₂₄ alkyl(hetero)aryl groups and C₇-C₂₄ (hetero)arylalkyl groups;
Y is N or CR¹⁵.

10. The method according to claim 1, wherein the click probe Q is selected from the group consisting of, optionally substituted, (hetero)cyclopropenyl group, (hetero)cyclobutenyl group, trans-(hetero)cycloheptenyl group, trans-(hetero)cyclooctenyl group, trans-(hetero)cyclononenyl group or trans-(hetero)cyclodecynyl group.

11. The method according to claim 10, wherein the click probe Q is selected from the group consisting of (Q40)-(Q50): 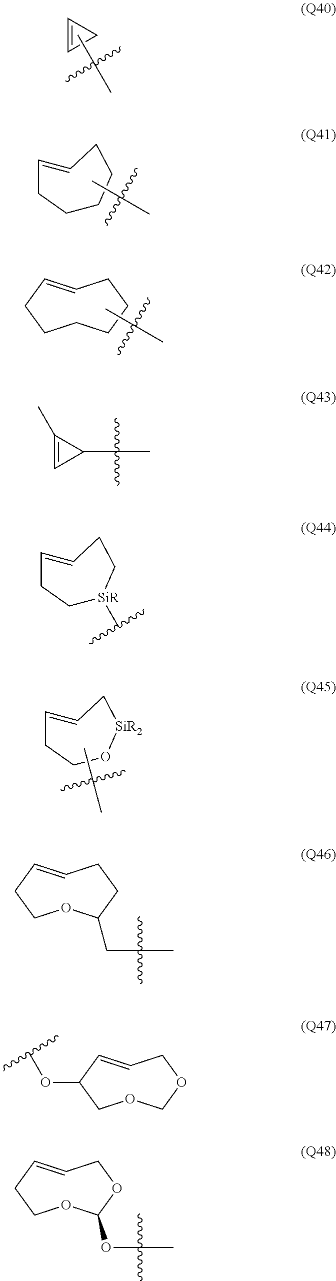 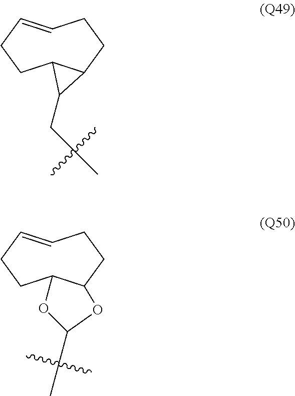 [SMILES: *CC1C2CC/C=C/CCC12 ; *C1OC2CC/C=C/CCC2O1]
wherein the R group(s) on Si in (Q44) and (Q45) is alkyl or aryl.

12. The method according to claim 1, wherein the payload D is a cytotoxin.

13. The method according to claim 12, wherein the cytotoxin is selected from colchicine, vinca alkaloids, anthracyclines, camptothecins, doxorubicin, daunorubicin, taxanes, calicheamycins, tubulysins, irinotecans, an inhibitory peptide, amanitin, deBouganin, duocarmycins, maytansines, auristatins, enediynes, pyrrolobenzodiazepines (PBDs) or indolinobenzodiazepine dimers (IGN) or PNU-159,682 and derivatives thereof.

14. The method according to claim 12, wherein the cytotoxin is selected from calicheamicin, PBD dimer, SN-38, MMAE or exatecan.

15. The method according to claim 1, wherein the biomolecule is selected from the group consisting of proteins, glycoproteins, antibodies, polypeptides, peptides, glycans, lipids, nucleic acids, oligonucleotides, polysaccharides, oligosaccharides, enzymes, hormones, amino acids and monosaccharides.

16. The method according to claim 15, wherein the biomolecule is selected from the group consisting of mAb, Fab, VHH, scFv, diabody, minibody, affibody, affylin, affimers, atrimers, fynomer, Cys-knot, DARPin, adnectin/centryin, knottin, anticalin, FN3, Kunitz domain, OBody, bicyclic peptides and tricyclic peptides.

17. The method according to claim 1, wherein the click probe F is connected to a monosaccharide moiety.

18. The method according to claim 17, wherein the click probe F is connected to a terminal monosaccharide moiety of a glycan of an antibody.

19. A method of preparing a bioconjugate having structure B—(Z—L—D),, wherein x payloads D are covalently attached to a biomolecule B via connecting group Z, the method comprising click reacting click probe Q with click probe F, wherein the reaction is between:
(i) an alkyne or alkene compound of structure Q—L—D, wherein
(a) Q is a click probe comprising a cyclic alkyne moiety or a cyclic alkene moiety,
(b) L is a linker, and
(c) D is a payload; with
(ii) a molecule of structure B—(F)x, wherein
(a) B is a biomolecule that is functionalized with x click probes F;
(b) F is a click probe capable of reacting with Q, and
(c) xis an integer in the range of 1-10.
